(19) United States
(12) Patent Application Publication
(10) Pub. No.: US 2013/0029424 A1
(43) Pub. Date: Jan. 31, 2013

(54) METHOD FOR THE GENERATION OF MONOCLONAL ANTIBODIES DERIVED FROM HUMAN B CELLS

(71) Applicants: Barton F. Haynes, Durham, NC (US); Mattia Bonsignori, Durham, NC (US); Kwan-Ki Hwang, Durham, NC (US); Hua-Xin Liao, Durham, NC (US); David C. Montefiori, Durham, NC (US); Micah A. Luftig, Durham, NC (US)

(72) Inventors: Barton F. Haynes, Durham, NC (US); Mattia Bonsignori, Durham, NC (US); Kwan-Ki Hwang, Durham, NC (US); Hua-Xin Liao, Durham, NC (US); David C. Montefiori, Durham, NC (US); Micah A. Luftig, Durham, NC (US)

(21) Appl. No.: 13/640,190

(22) PCT Filed: Apr. 11, 2011
(86) PCT No.: PCT/US11/00645

§ 371 Date: Oct. 9, 2012

Related U.S. Application Data
(60) Provisional application No. 61/322,725, filed on Apr. 9, 2010.
(60) Provisional application No. 61/322,821, filed on Apr. 10, 2010.

(51) Int. Cl. C12N 15/86

(52) U.S. Cl. 435/467

(57) ABSTRACT

The present invention relates, in general, to human B cells, and, in particular to a method of immortalizing and cloning human B cells and to monoclonal antibodies derived therefrom. The invention further relates to methods of using the monoclonal antibodies for therapeutic and diagnostic purposes.

This application claims priority from U.S. Provisional Application No. 61/322,725, filed Apr. 9, 2010 and U.S. Provisional Application No. 61/322,821 filed Apr. 10, 2010, the entire contents of which are incorporated herein by reference.

This invention was made with government support under Grant No. AI067854-02 awarded by the National Institutes of Health. The government has certain rights in the invention.

### TECHNICAL FIELD

The present invention relates, in general, to human B cells, and, in particular to a method of immortalizing and cloning human B cells and to monoclonal antibodies derived therefrom. The invention further relates to methods of using the monoclonal antibodies for therapeutic and diagnostic purposes.

### BACKGROUND

The isolation and characterization of monoclonal antibodies that neutralize, for example, a broad spectrum of HIV-1 isolates are important to the design of an effective HIV-1 vaccine. An efficient method of obtaining such antibodies has, however, been elusive.

Epstein Barr Virus (EBV) has been used to immortalize human B cells that produce neutralizing antibodies (Traggiai et al, Nat. Med. 10(8):871 (2004) Epub 2004 Jul. 11; Corti et al, PLoS ONE 5:e8805 (2010)). The efficiency of immortalization, however, can be low, as can the cloning efficiency of the immortalized B cells.

The present invention provides a method for the rapid and efficient expansion of clonal memory B cells, for the isolation of antibody variable heavy (VH) and variable light (VL) genes and for the capture and growth of B cells producing broadly neutralizing antibodies (e.g., broadly neutralizing anti-HIV-1 antibodies).

### SUMMARY OF THE INVENTION

The present invention relates to human B cells. More specifically, the invention relates to a method of immortalizing and cloning human B cells, to monoclonal antibodies derived from such cells, and to nucleic sequences encoding VH and VL chains of the monoclonal antibodies. The invention further relates to methods of using the monoclonal antibodies for therapeutic and diagnostic purposes.

Objects and advantages of the present invention will be clear from the description that follows.

### BRIEF DESCRIPTION OF THE DRAWINGS

FIG. 1. Total IgG and IgM levels in the culture supernatants of EBV-transformed IgG+ memory B cells isolated from an uninfected PBMC sample. After EBV infection, the cells were plated at 30 cells/well and incubated in the presence of ODN2006+Chk2 inhibitor or IL-2+R848 (Table III) for 14 days. The data are expressed in ng/ml.

FIG. 2. Total IgG levels in the culture supernatants of EBV-transformed IgG+ memory B cells isolated from an uninfected PBMC sample. After EBV infection, the cells were plated at 10 or 30 cells/well and incubated in the presence of ODN2006+Chk2 inhibitor or IL-2+R848 (Table VI) for 14 days. The data are expressed in ng/ml. (FIG. 2 includes graphs at 3 and 1 cell/well.)

FIG. 3. Summary of method for highly efficient and high throughput operation of monoclonal antibodies for IgG+ memory B cells.

FIG. 4. Isolation of HIV+broad neutralizing human monoclonal antibodies from CHAVI 008 patients.

FIG. 5. Clonal relationship of neutralizing antibodies derived from CH0219.

FIG. 6. Variable heavy (VH) and variable light (VK) chains gene sequences and amino acid sequences of antibodies 1-27-G2, 1-19-F10 and 1-27-G11 derived from CH0219.

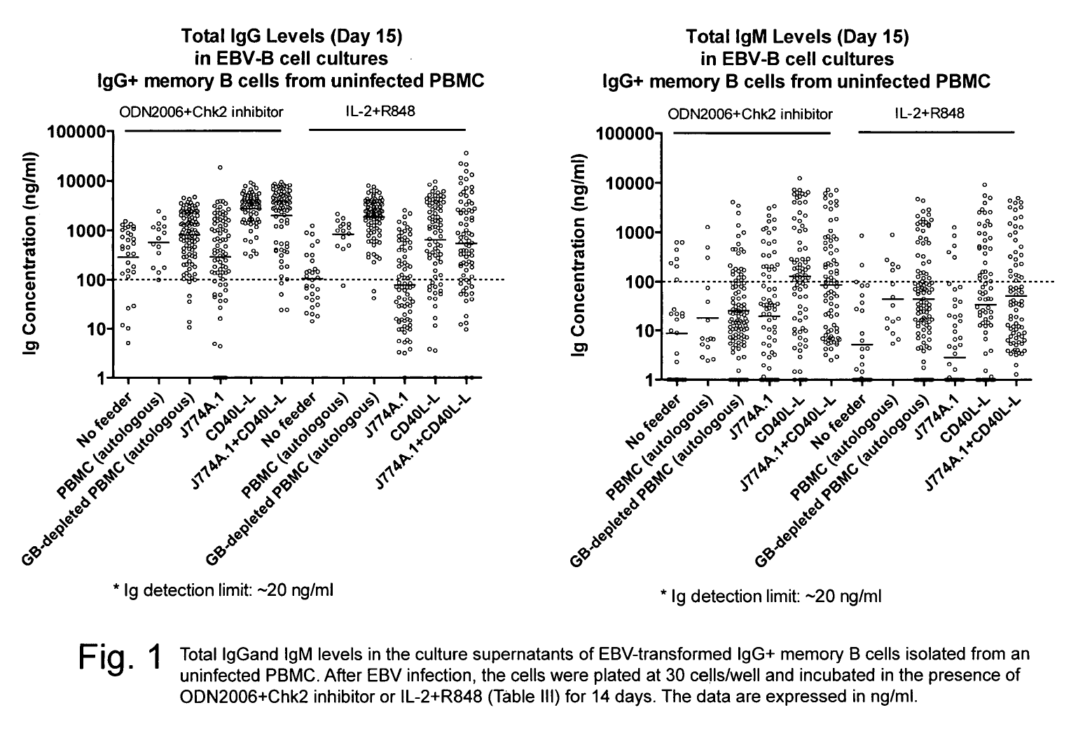

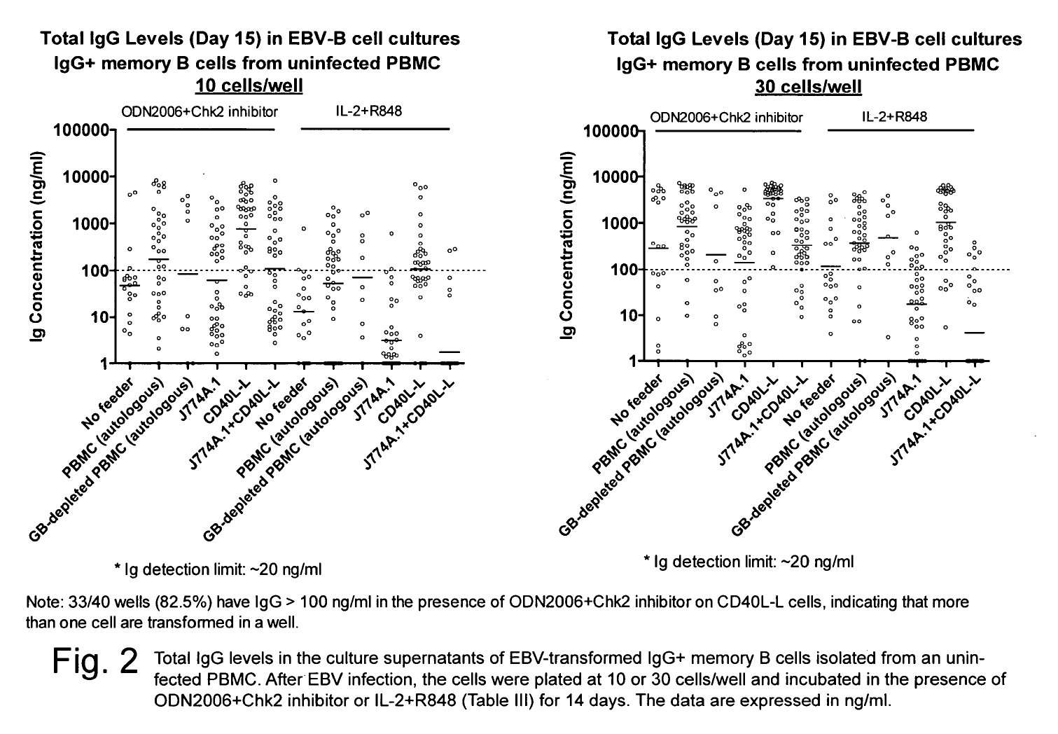

### DETAILED DESCRIPTION OF THE INVENTION

The present invention relates, at least in part, to a method of producing immortalized B cells. The method can comprise transforming B cells using EBV using standard protocols. While the Example below involves the use of IgG+ memory B cells obtained from peripheral blood, the method can be applied to other B cell subsets from other tissues (for example, from mucosal or lymphoid tissue) and to cells of other isotypes (for example, but not limited to, IgM+ naive B cells or IgA-memory B cells). The present method couples CD40 ligation and EBV transformation with TLR-9 ligation. This approach makes possible the rapid and efficient screening of large numbers of B cells.

In accordance with the invention, B cells transformed with EBV can be expanded in culture and the culture supernatant screened for the presence of antibodies having a desired antigen specificity (e.g., the antigen specificity of the antibody can be directed against a pathogen (e.g., HIV-1 or other pathogen referenced in PCT/US09/63271), chemical or toxin). Immortalized clones of the antibody-producing B cells can then be isolated and further cultured under conditions such that the antibodies having the desired antigen specificity are expressed. The antibodies can be isolated from the culture medium using standard techniques. The B cells can be expanded monoclonally, oligoclonally or polyclonally by varying the cell density in the cultures (for example, from cell densities of 10 or less cells/well for a predominantly monoclonal expansion to 100 or more cells/well for polyclonal expansion). Advantageously, the cells are seeded at a density of <10 cells/well.

Nucleic acid sequences encoding the monoclonal antibody (or VH and/or VL chains thereof) can be isolated from the cloned B cells using standard techniques.

In accordance with the invention, host cells (e.g., 293T cells) can be transfected with a construct comprising the nucleic acid sequences encoding VH and/or VL chains of the monoclonal antibody of interest under conditions such that those sequences are expressed and antibodies having the desired specificity are produced. Thus produced antibodies can be isolated (e.g., from the host cell or from media in which the host cells are cultured) using standard techniques.

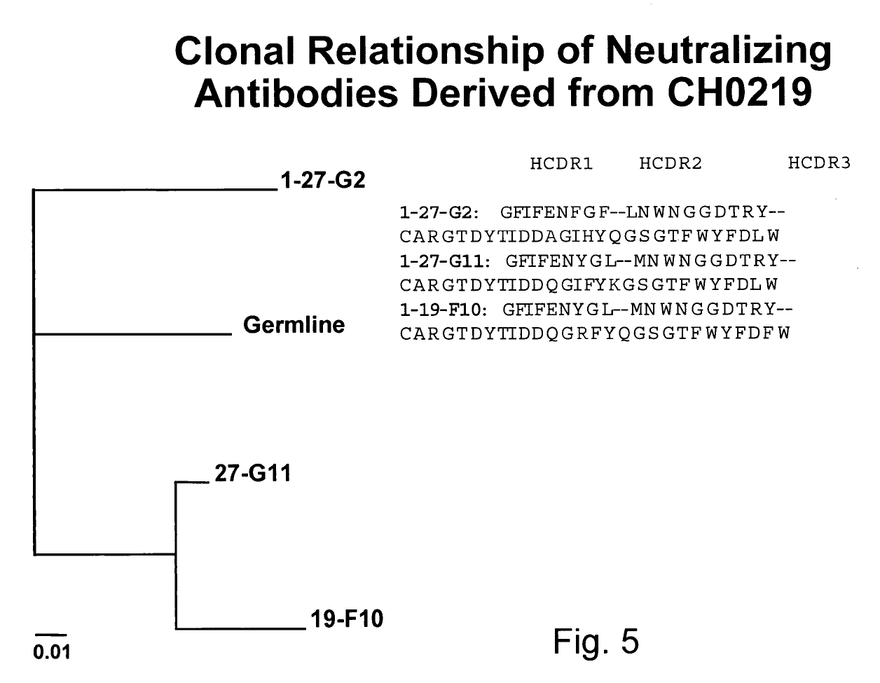

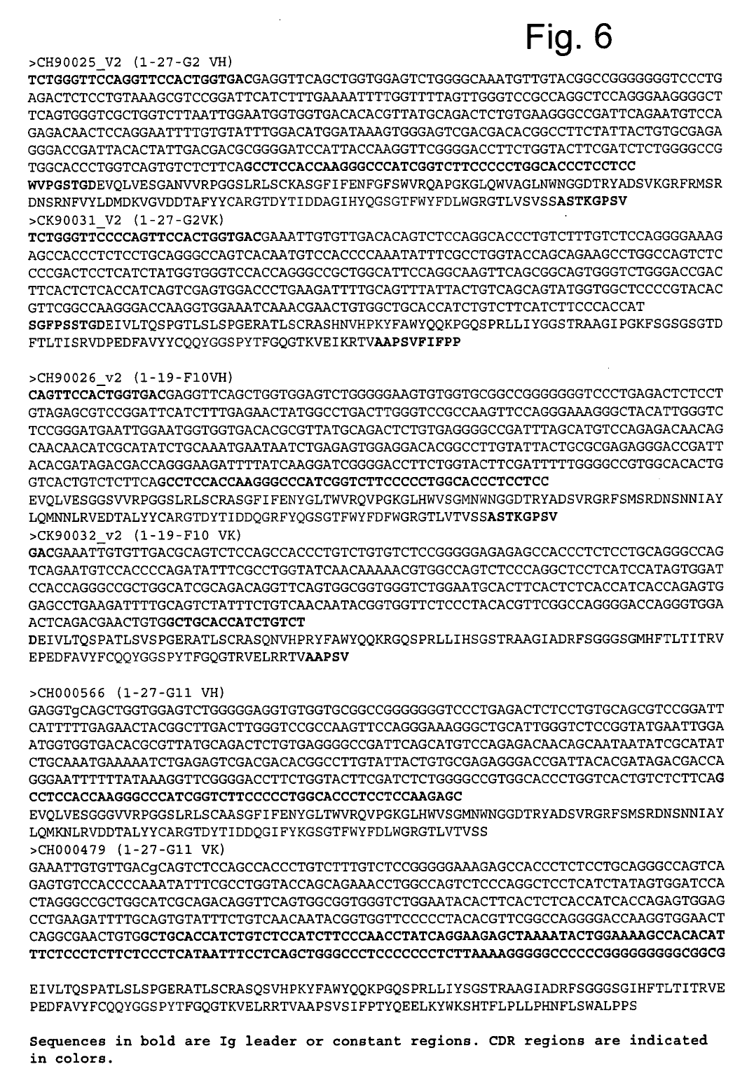

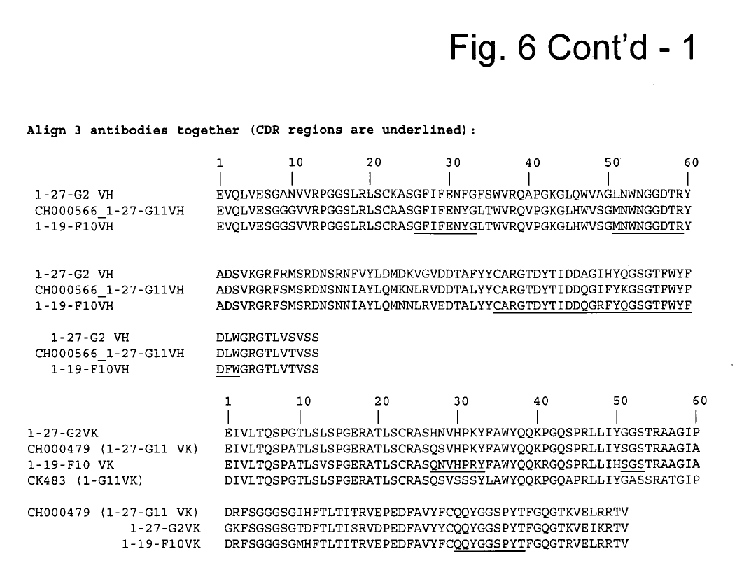

Preferred antibodies of the invention are derived from CH0219 and include 1-27-G2, 1-27-G11 and 1-19-F10 (see FIGS. 5 and 6).

The antibodies of the present invention can be used, for example, diagnostically or therapeutically. For example, the antibodies can be useful in the identification and/or purification (e.g., using affinity purification techniques) of an individual polypeptide or other antigen against which they are directed. The antibodies can also be employed as reagents in, for example, immunoassays, radioimmunoassay (RIA) or enzyme-linked immunosorbent assays (ELISA). The antibodies can be labeled with a detectable label (such as a radioisotope, a fluorescent molecule or an enzyme). Antibodies produced by the methods disclosed herein can be used for detecting pathogens, such as HIV-1.

The invention also includes pharmaceutical compositions comprising antibodies of the present invention and a carrier. In some embodiments, pharmaceutical compositions comprising transformed and/or activated B cells of the presently disclosed invention are provided. Pharmaceutical compositions can also contain a pharmaceutical acceptable carrier or adjuvant.

The antibodies of the present invention, or fragments (e.g., antigen binding fragments) thereof, can be used for the treatment of disease, for the prevention of disease and/or for the diagnosis of disease. In some embodiments, the monoclonal antibodies of the presently disclosed subject matter can be administered to a subject in need thereof, in a pharmaceutical composition or medicament as described above.

Suitable routes of administration will depend on the disease to be treated, prevented or diagnosed but can include IV, IM, intra-nasal or subcutaneous. Administration directly to mucosal tissues can also be effected, when appropriate.

Certain aspects of the present invention are described in greater detail in the Example that follows. In the study of the Example, CD40L-expressing L cells were used, however, other CD40L-expressing cells (e.g., CD40L-expressing 293T cells, CD4⁺ T cells, or macrophages, dendritic cells, FDCs, thymic epithelial and endothelial cells), anti-CD40 antibodies (Galibert et al, Eur. J. Immunol. 25(3):733-737 (1995), Saeland et al, J. Exp. Med. 178(1):113-120 (1993), Bamchereau and Rousset, Nature 353(6345):678-679 (1991)) or other means for inducing CD40 ligation can be used (e.g., CD40 agonists). While the strain of EBV used in the Example was B95-8, other strains of EBV can also be used, as can other viruses that mediate comparable effects (e.g., Herpesvirus papio for monkeys). The TLR ligand used in the Example was ODN2006 (Traggiai et al, Nat. med. 10(8):871 (2004)) but the invention includes the use of other TLR-ligands (e.g., LPS, R848). A Chk2 kinase inhibitor was also used in the Example. Other anti-apoptotic agents can also be employed (e.g., ATM inhibitor or any inhibitor of oncogenic stress or DNA damage response). It will be appreciated that the stimulation cocktail used gave better yields than other stimulation regimens.

As described in the Example below, IgG⁺ memory B cells from peripheral blood were enriched through a two-step process: (1) depletion of CD2+, CD14+, CD16+, CD235a+ and IgD+ cells through magnetic bead separation using a cocktail of PE-conjugated antibodies as primary antibodies and commercially available anti-PE microbeads as secondary antibodies; (2) enrichment of IgG+ cells through positive selection with an anti-IgG microbead-conjugated antibody applied on the negatively selected cell fraction. Cells were stimulated for 14 days. At the end of the stimulation, cells are assayed in bulk for RNA-extraction and/or preserved in RNAlater for later treatment. A determination was made of the cell dilution that resulted in a monoclonal expansion according to the single-hit model of Poisson distribution in repeated experiments on HIV-1 chronic and uninfected subjects. The statistical model was validated by sequencing the VH chains of 4 selected wells both from fresh and in RNAlater-treated stimulated cells.

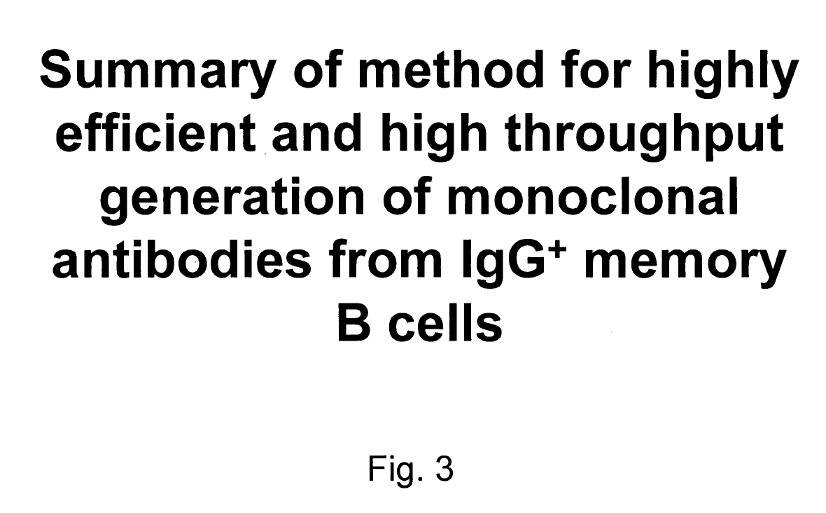

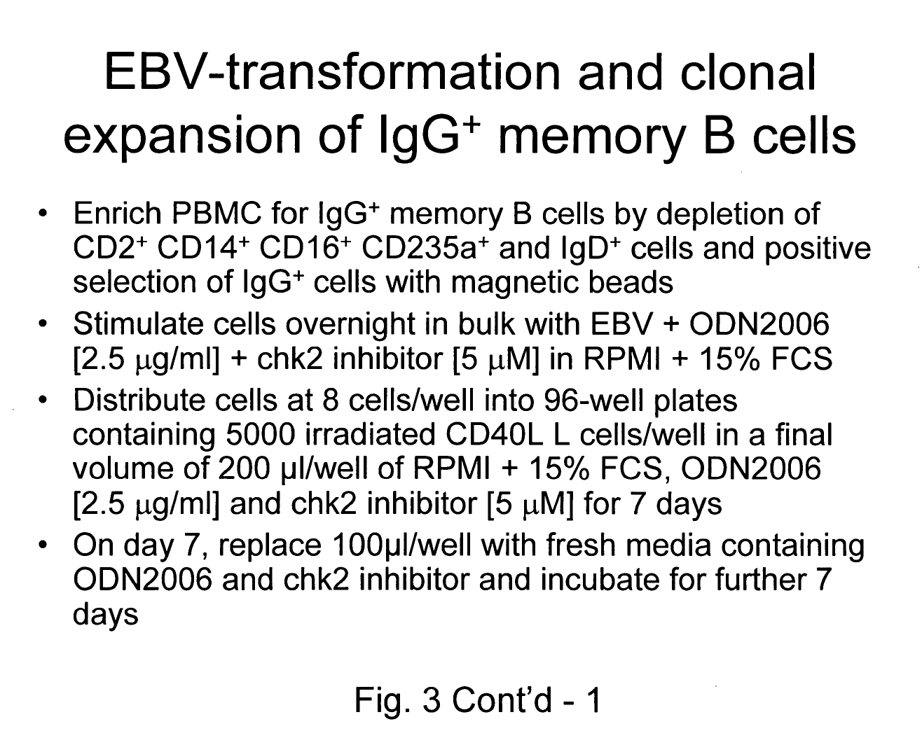

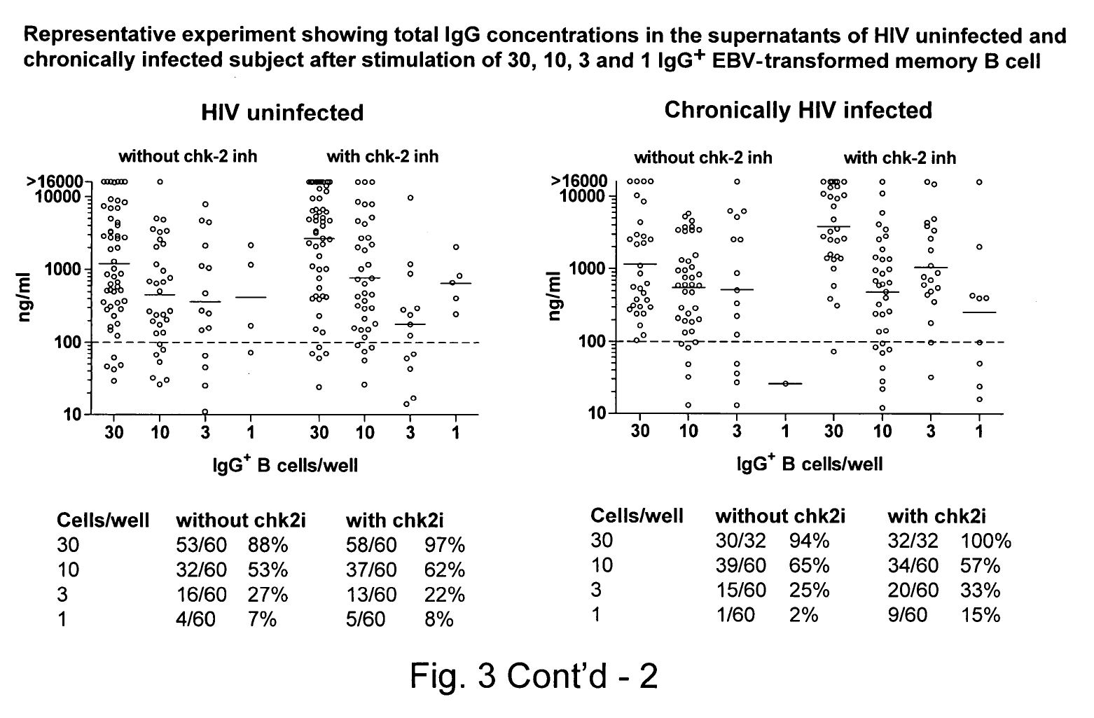

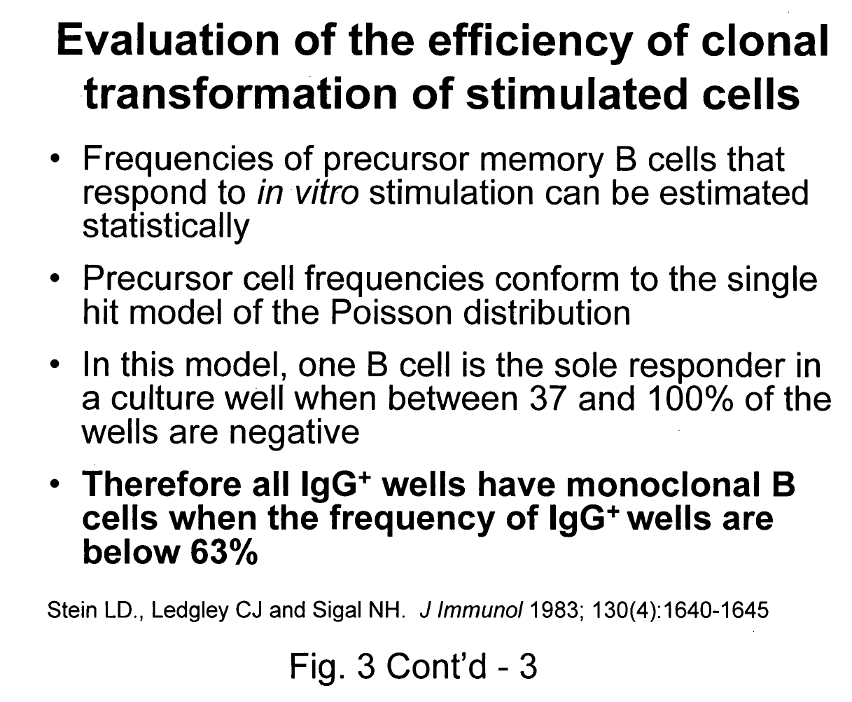

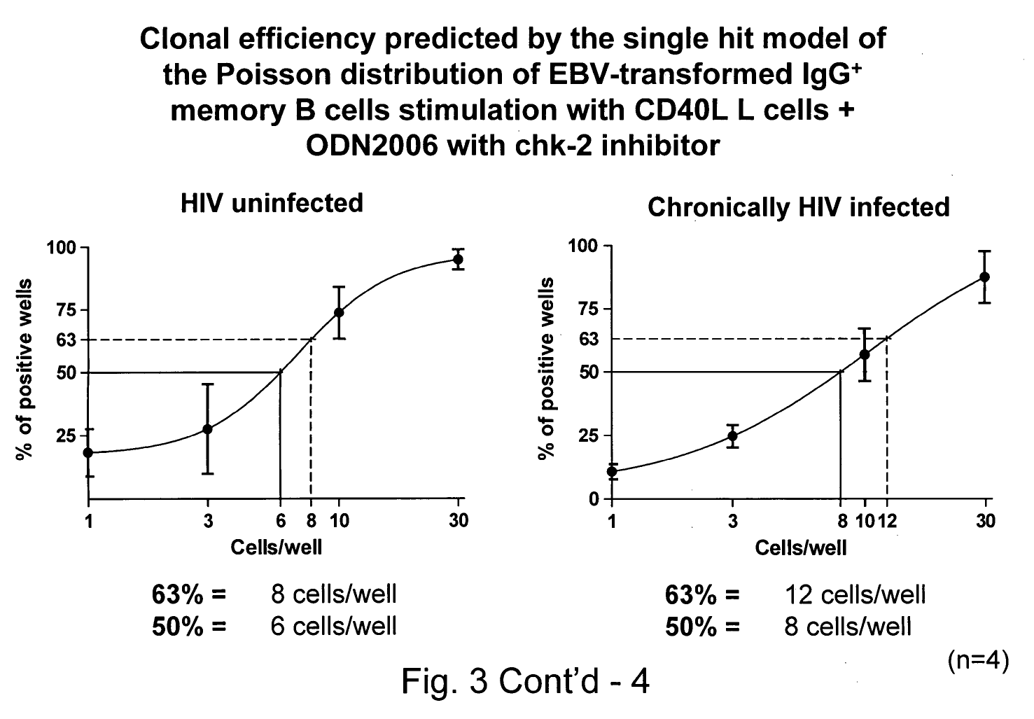

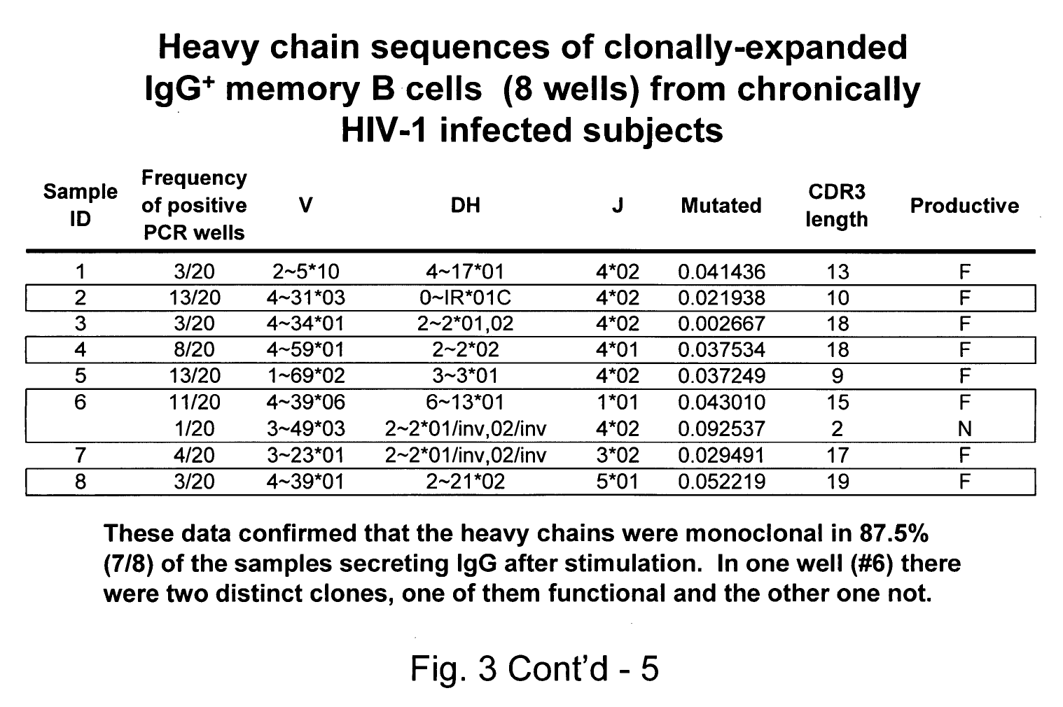

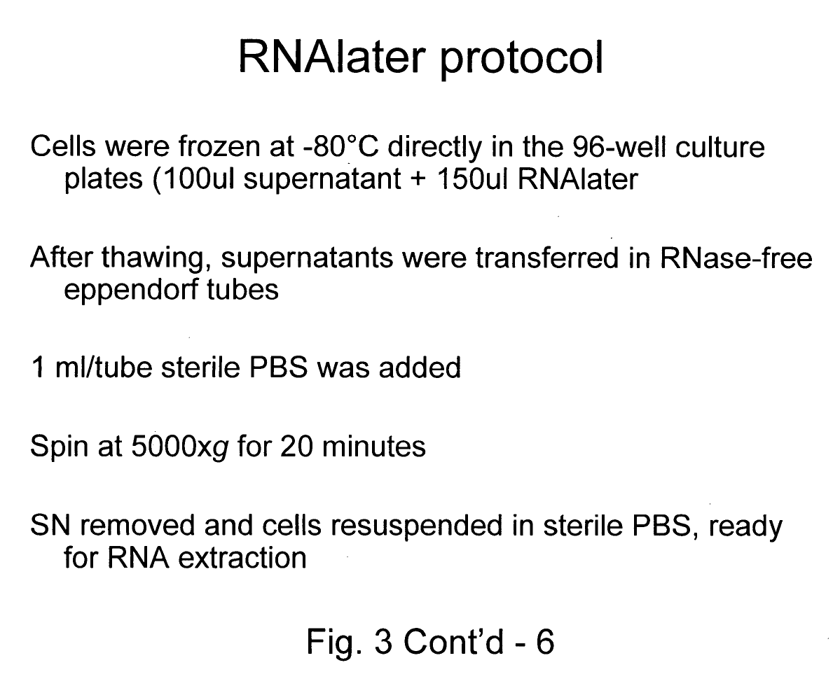

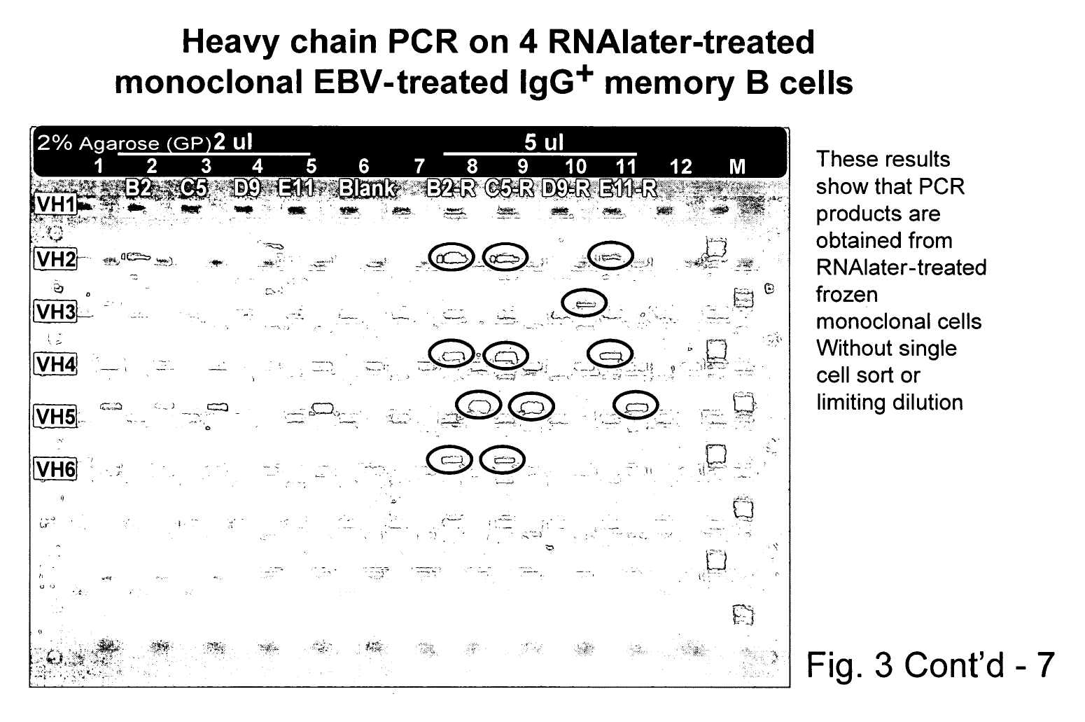

The results are summarized in FIG. 3. The single hit model of the Poisson distribution predicted that in chronically HIV-1 infected subjects, monoclonal B cell expansion occurs when 10 or less cells/well are put in culture. PCR data confirmed that, at 10 cells/well, heavy chains were monoclonal in 87.5% (⅞) of the IgG+ samples secreting IgG after stimulation. In one well there were two distinct clones, one of them functional and the other one not. To account for patient-to-patient variability, the decision was made to plate cells at a density of 8 cells/well (50% of positive wells predicted with the single hit model of the Poisson distribution) to increase the chances of growing monoclonal cultures.

The methods disclosed herein can be practiced in connection with human and non-human mammals (and, as appropriate, cells (e.g., B-cells) derived therefrom), including primates, rats, mice, guinea pigs, rabbits, hamsters, domestic animals (e.g., dogs and cats) and farm animals (e.g., cows, pigs, horses).

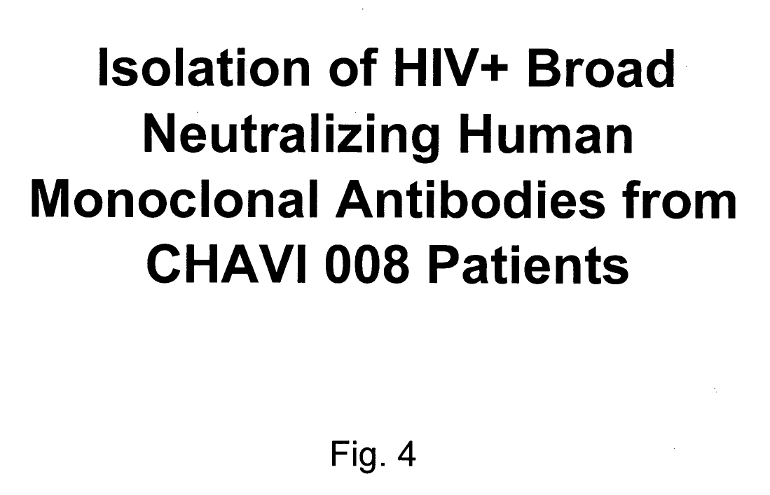

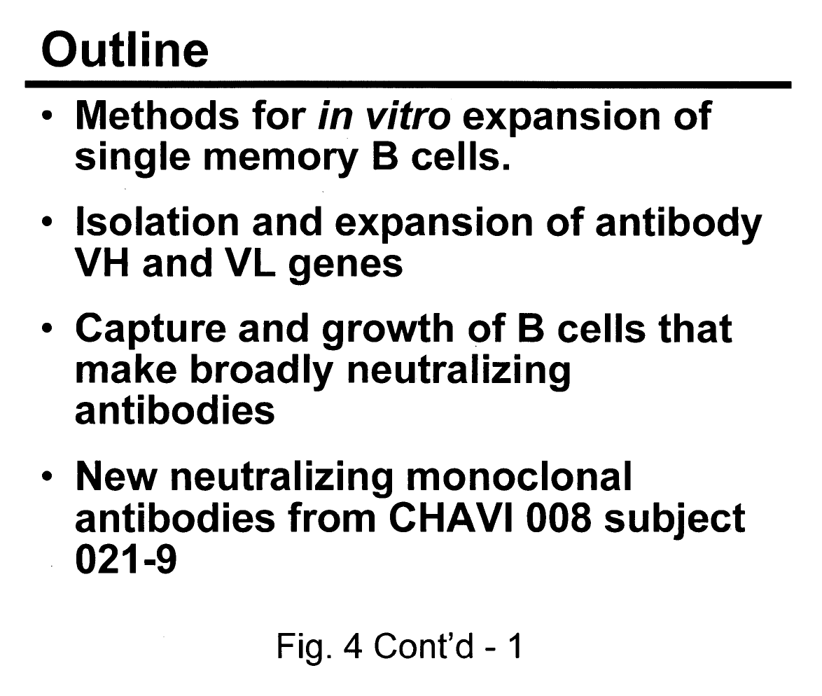

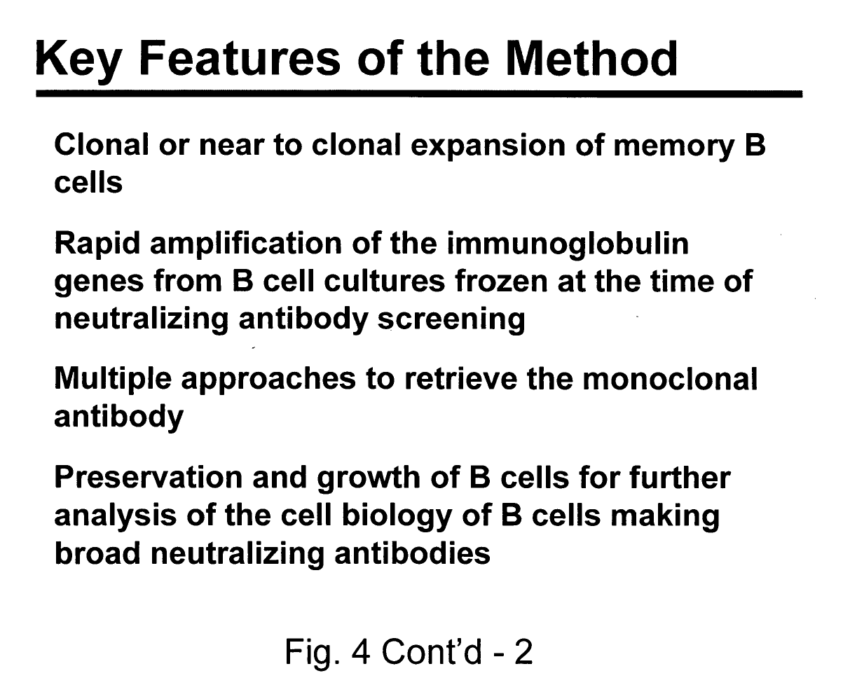

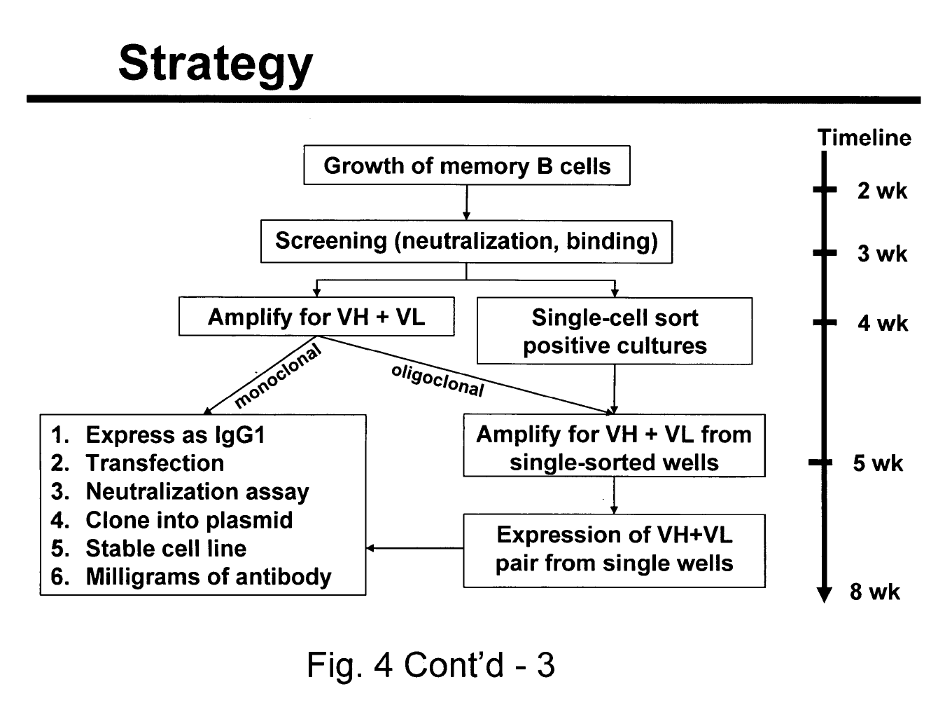

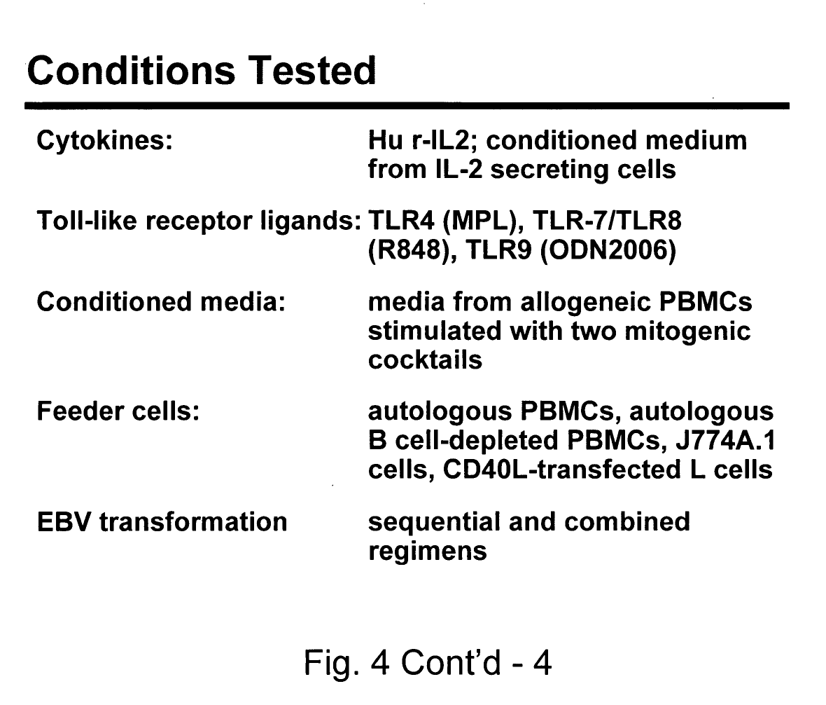

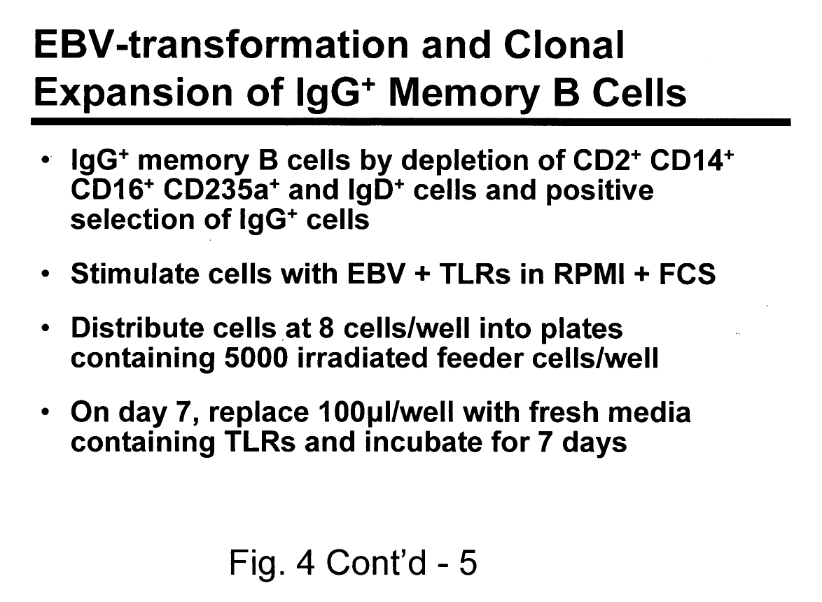

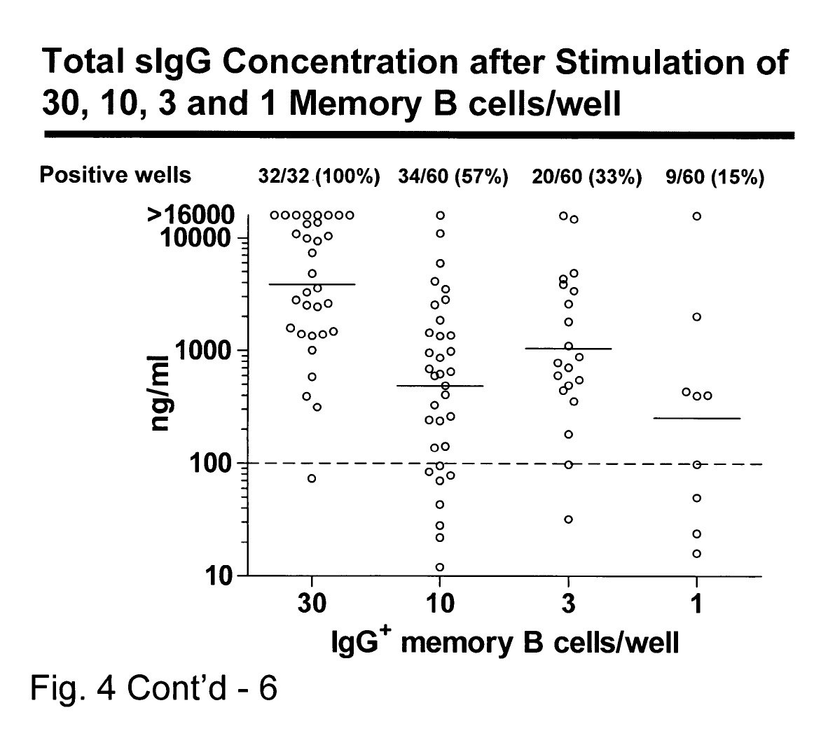

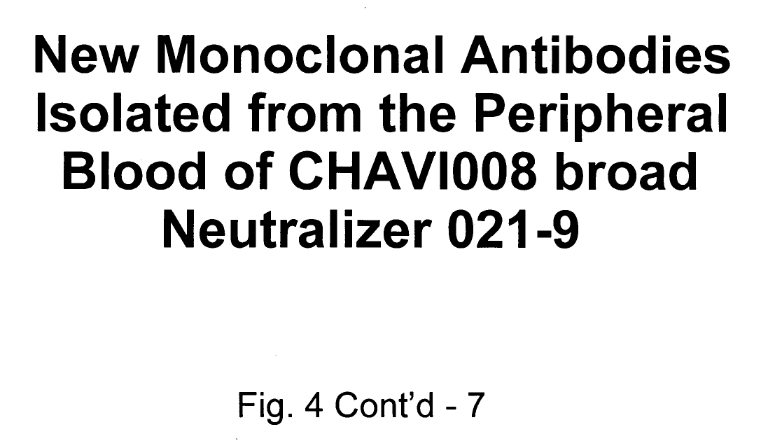

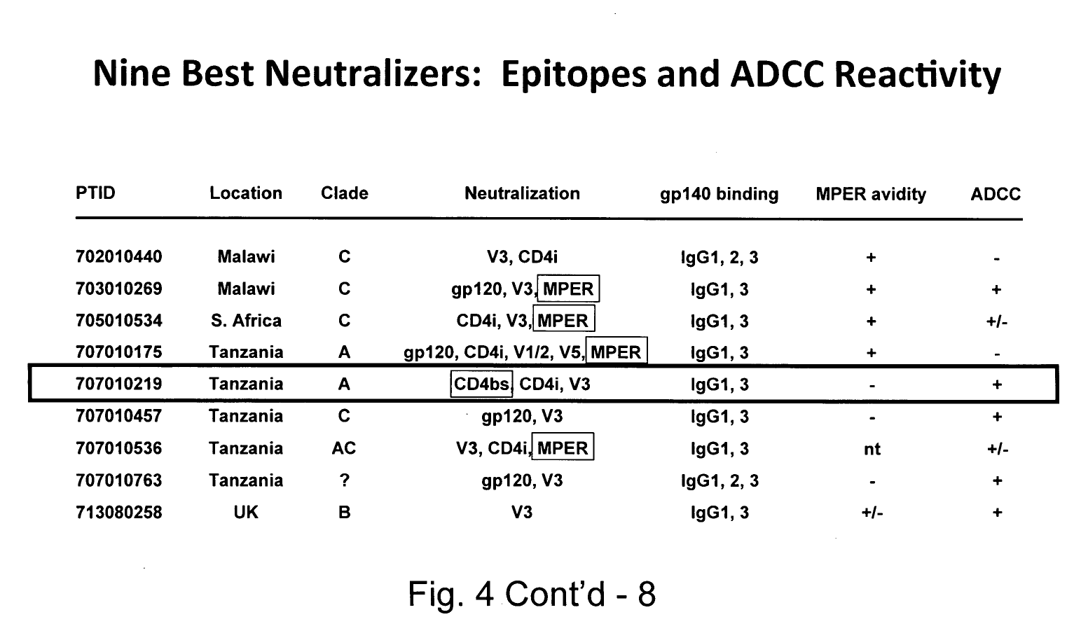

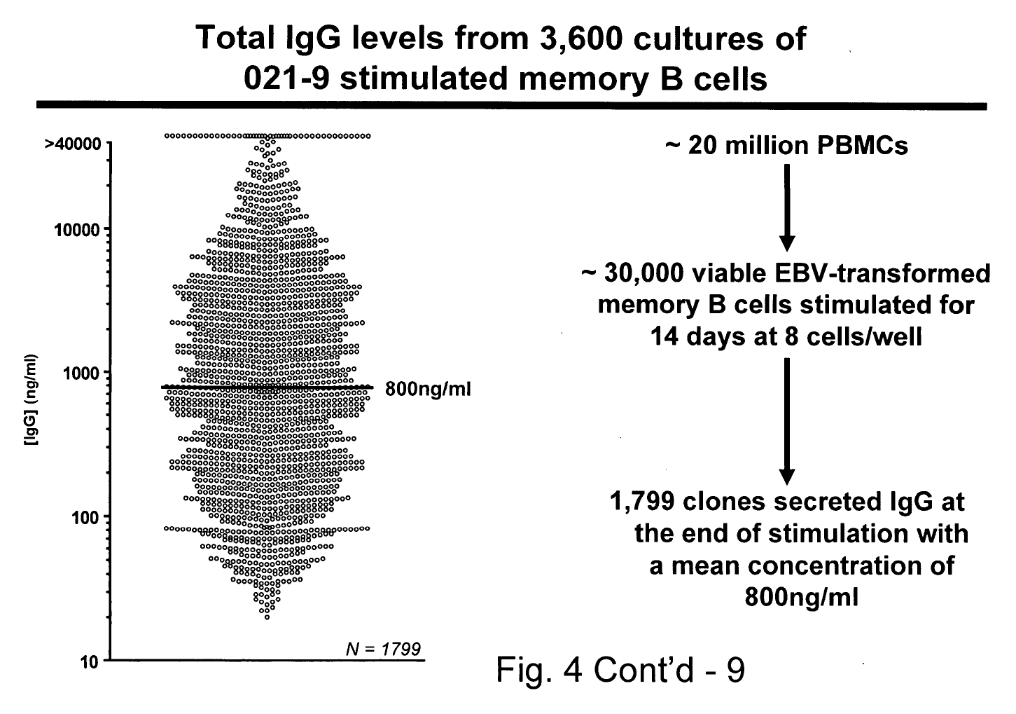

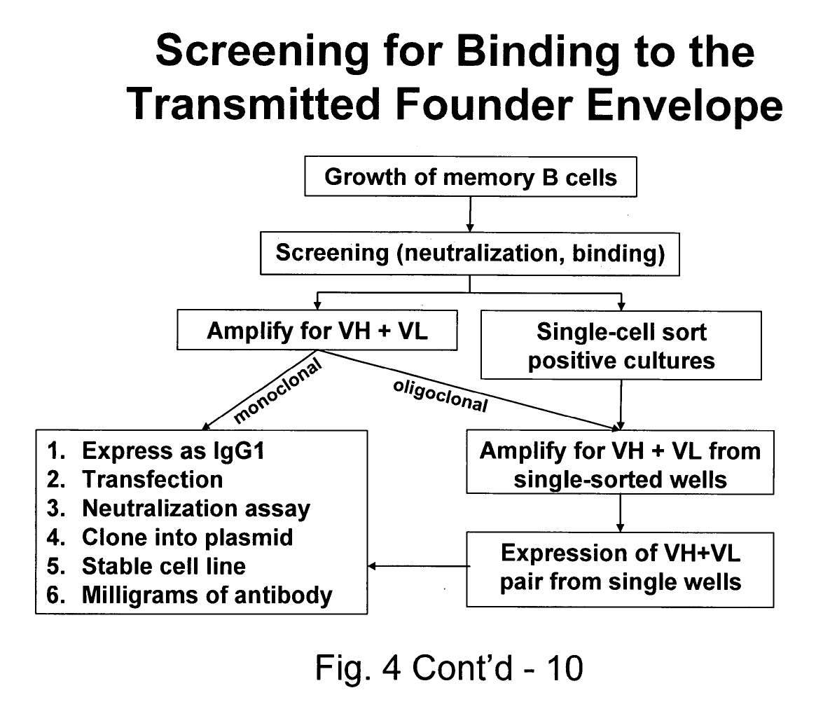

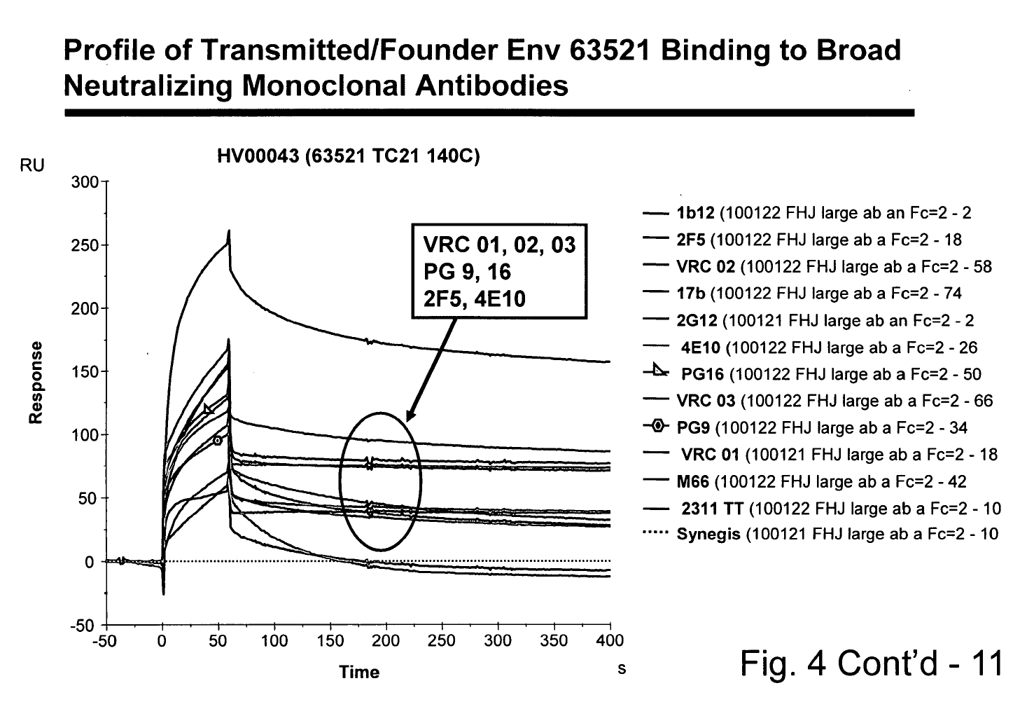

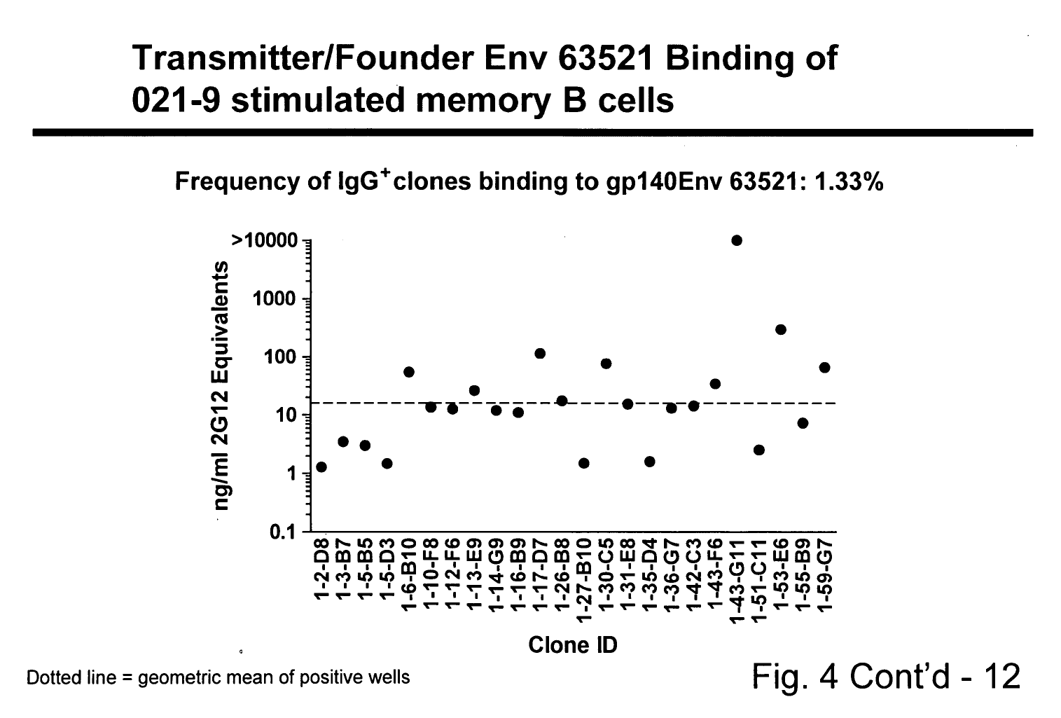

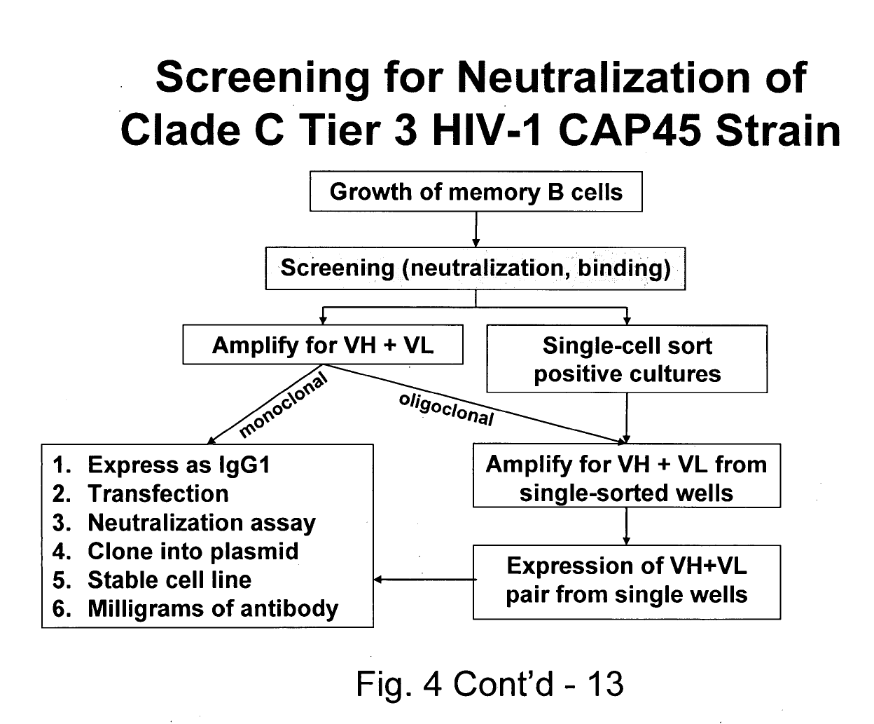

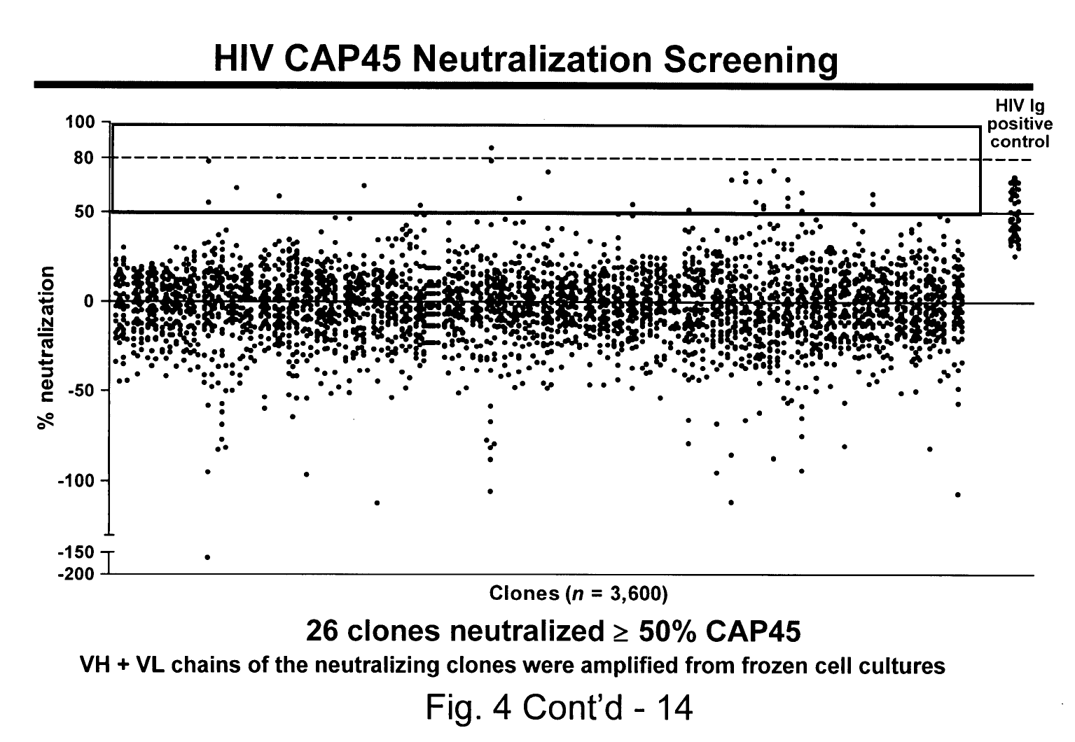

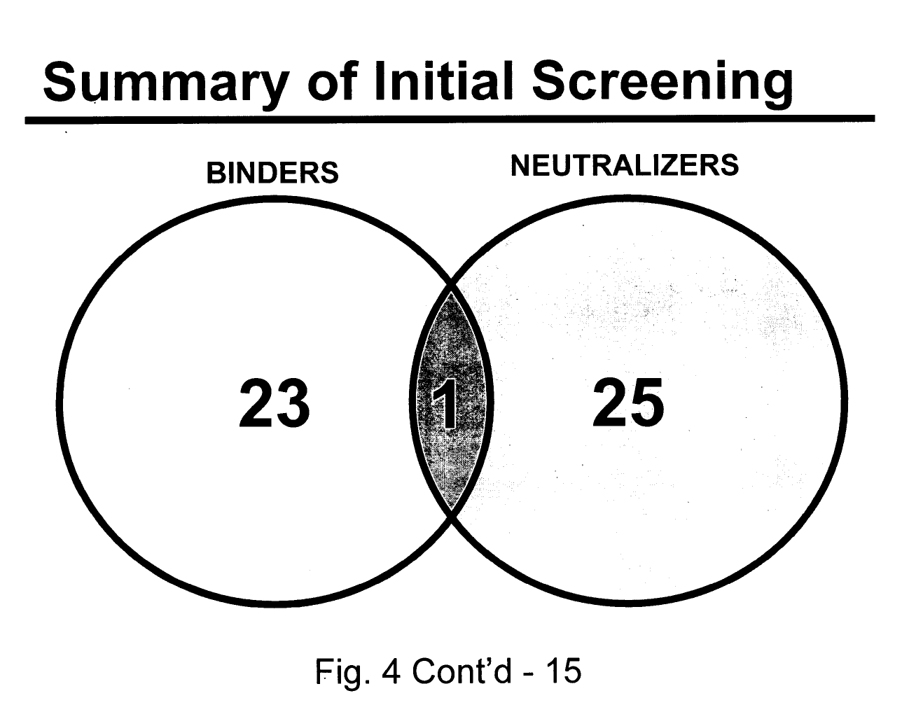

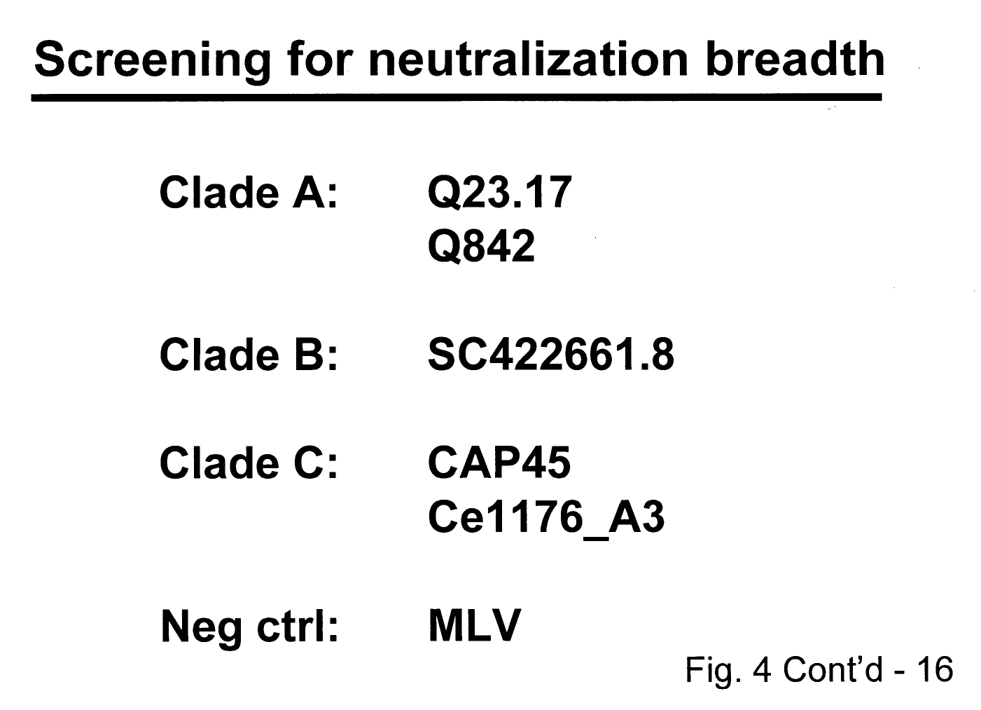

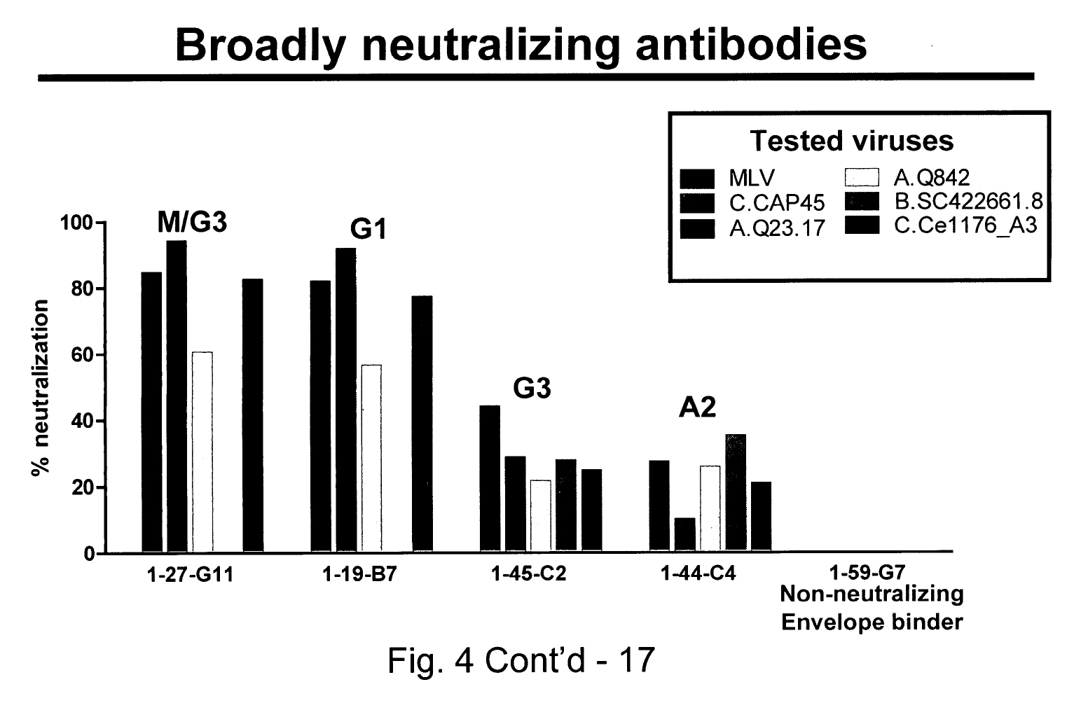

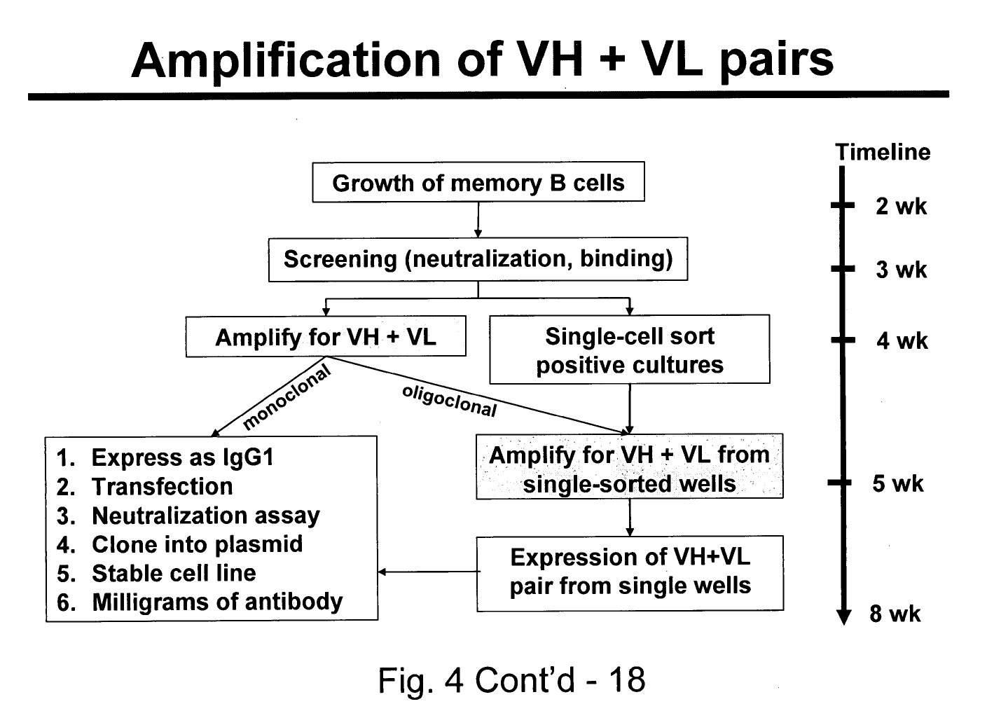

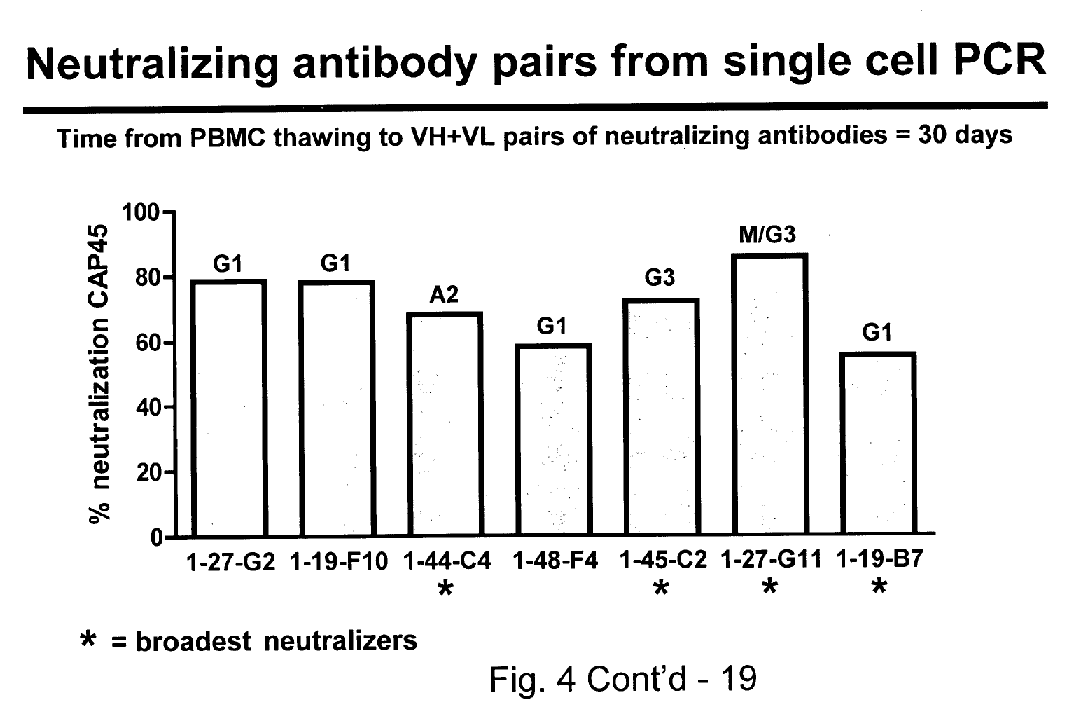

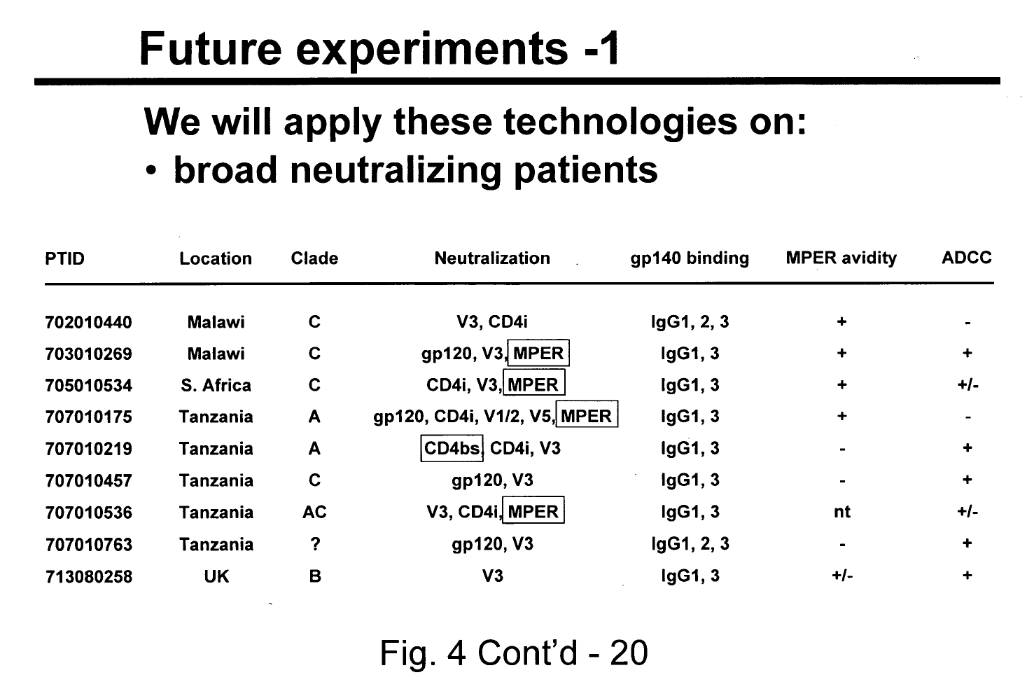

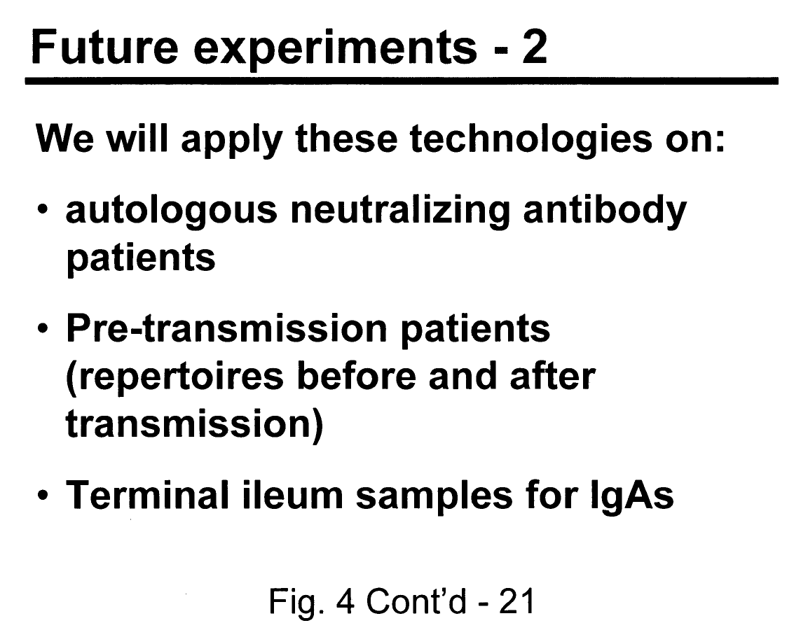

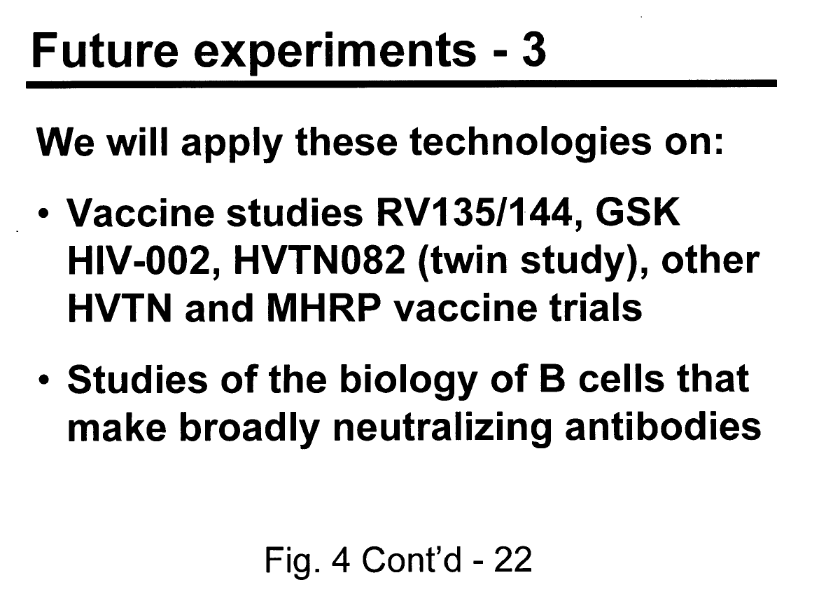

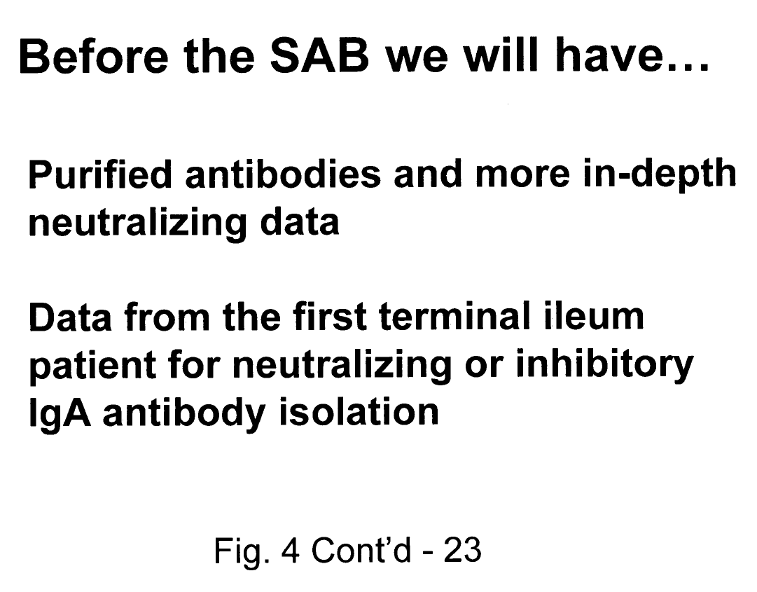

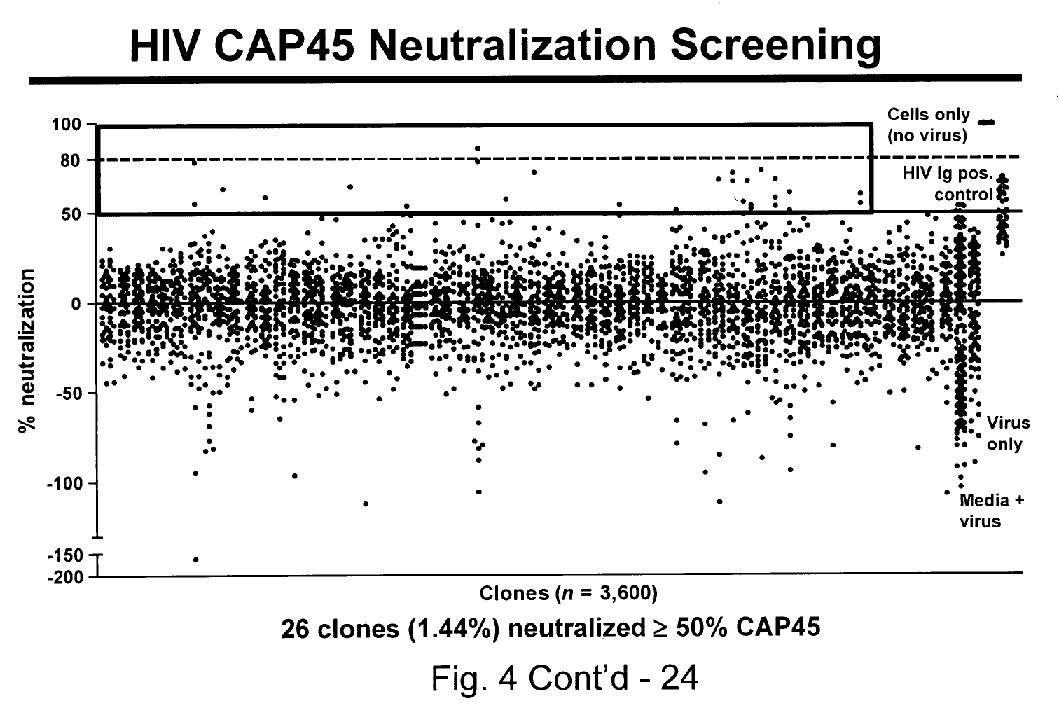

Certain aspects of the invention are described in greater detail in FIGS. 3 and 4 (see also PCT/US09/63271, filed Nov. 11, 2009, the entire content of which is incorporated herein by reference). (Incorporated by reference are U.S. Provisional Appln. No. 61/322,663 and 61/322,725, both filed Apr. 9, 2010.)

### EXAMPLE

### Experimental Details

### Preparation of Complete Medium

Complete Medium (CM) for PBMC, EBV-B cells, J774A.1, and K6H6/B5 cell lines

RPMI 1640 (Invitrogen) supplemented with:
1) 15.2% heat-inactivated fetal calf serum (FCS)
2) 1% non-essential amino acids (NEAA)
3) 1 mM sodium pyruvate
4) 15 mM HEPES buffer, pH 7.3
5) 2 mM L-glutamine (Glu)
6) 100 U/ml penicillin G (Pen)
7) 100 μg/ml streptomycin (Strep)

Preparation:
1. Thaw a bottle of FCS (500 ml/bottle, −20° C.) and incubate at 56° C. for 30 min for heat inactivation (inactivation of c′)
2. Let bottle cool down and add 31 ml of Glu+Pen/Strep (100×, 100 ml/bottle, −20° C.)
3. Aliquot the FCS-antibiotics mixture into tubes at 50 ml/tube and freeze at −20° C.
4. Add 100 ml of FCS-antibiotics mixture to bottle of RPMI 1640 (500 ml/bottle, 4° C.)
5. Add 6.2 ml of HEPES buffer (100×, 100 ml/bottle, 4° C.) into the bottle
6. Add 6.2 ml of sodium pyruvate (100×, 100 ml/bottle, 4° C.)
7. Add 6.2 ml of NEAA (100×, 100 ml/bottle, 4° C.)
8. Filter the CM through a 0.22 μm filter unit.
9. Total CM: 618.6 ml

### IgG⁺ Memory B Cell Stimulation Protocol

Thaw frozen PBMCs as per standard operating procedure
Resuspend PBMCsat 10×10⁶ PBMC/ml in PBS/1% BSA and round up to the higher 100 μl. Keep cells at 4° C.
Prepare a master mix of the following PE-conjugated antibodies at the following concentrations (determined by titration of each single lot):
   Anti-CD235a [0.08 μl/10⁶ PBMCs]
   Anti-CD2 [5 μl/1⁰⁶ PBMCs]
   Anti-CD16 [5 μl/10⁶ PBMCs]
   Anti-IgD [5 μl/10⁶ PBMCs]
   Anti-CD14 [20 μl/1⁰⁶ PBMCs]
Aliquot 100 μl cells in 5 ml round bottom FACS tubes and split equal volumes of master mix in each tube
Mix and incubate 30 mins at 4° C.
Add 2 ml/tube PBS/1% BSA
Centrifuge 1500 rpm 5 mins
Remove supernatant without disturbing the pellet
Add 100 μl/tube PBS/1% BSA
Add 200 μl/tube anti-PE Microbeads
Mix and incubate at 4° C. for 15 mins
Add 2 ml/tube PBS/1% BSA
Centrifuge 300×g for 10 mins
Remove supernatant without disturbing the pellet
Tap well the tube to loosen up the pellet to prevent cell clumps. Add 1 ml PBS/1% BSA/tube and mix well. Visually check for cell clumps. In case plumps are present, break them by mixing with a P1000
Transfer tubes into the AutoMACS chiller for 5 ml tubes
Run DEPLETES program

### PROCESSING THE B CELL FRACTION

Collect the flow-through tubes
Combine the cells together in a 15-ml conical tube(s)
Centrifuge 300×g 10 mins
Resuspend in 200 μl PBS/1% BSA.
Add 20 μl anti-IgG Microbeads (for up to 10⁷ cells)
Mix well and incubate for 15 minutes at 4° C.
Add 2 ml PBS/1% BSA
Centrifuge 300×g 10 minutes
Resuspend in 1 ml PBS/1% BSA
Transfer tube in 15-ml tube chiller
Run POSSEL_S program
Collect the positively selected fraction
Centrifuge 1500 rpm 5 mins
Remove SN without touching the bottom
Resuspend in complete medium
Determine accurate cell count and volume
Stimulate with EBV suspension (B95-8; 1 ml/100,000 B cells), ODN2006 (2.5 μg/ml) and chk2-inhibitor (5 μM) overnight
Meanwhile, γ-irradiated (75 Gy) CD40L-transfected L cells are plated overnight into 96-well tissue culture plates at 5,000 cells/well
After overnight incubation, check EBV-transformed cell viability
Resuspend cells at 80 viable cells/ml in complete media containing ODN2006 and chk2-i and transfer 100 μl/well (i.e. 8 cells/well, which equals 1 activated cell/well for chronically HIV-1 infected subjects)
Aliquot 100 μl/well of cell suspensions in 96-well plate containing 5000 CD40L L cells in 100 μl/well, one plate/cell concentration (total 6 plates, i.e. 3 plates/cell aliquot)
Incubate at 37° C. for 14 days

### EBV Transformation of B Cells

For IgG Memory B Cells
1) On the day before EBV infection, prepare irradiated CD40L-L cells (7,500 cGray) and distribute the cells into 96-well plates (round-bottom) at 5,000 cells/well (100 μl/well).
2) Centrifuge memory B cells in complete medium (CM) at 1200 RPM (300 g) for 5 min at 4 ° C.
3) Resuspend the pellet in 1 ml of CM (1×10⁵ cells/ml) containing ODN 2006 (5.0 μg/ml) and Chk2 inhibitor II (10 μM).
4) Add EBV suspension (B95-8 cell supernatant*, stored in a −80° C. freezer or a liquid N₂ tank) at 1 ml/1×10⁵ memory B cells.
5) The final concentrations of ODN 2006 and Chk2 inhibitor II are 2.5 μg/ml and 5 μM, respectively.
6) Incubate the cells overnight at 37° C. in a 5% CO₂ incubator.
7) After incubation, count the viable cells and resuspend them at 80 viable cells/ml in CM containing 5 μg/ml PS2006 (ODN 2006) and 10 μM Chk2 inhibitor II.
8) Distribute the cells at 8 cells/well (100 μl/well) into the 96-well plates with the irradiated CD40L-L cells.
9) Incubate the cells at 37° C. in a 5% CO₂ incubator.
10) On Day 7, take 90 μl of supernatant from each well and replace with 100 μl of fresh CM containing 2.5 μg/ml PS2006 (ODN 2006) and 5 μM Chk2 inhibitor II.
11) On Day 14, collect 100 μl of supernatant from each well for functional assays.
12) Collect additional 20 μl of supernatant from each well for binding assays.
13) Mix and collect 30 μl of the cells from each well for a storage in RNA Later.
14) Add 160 μl of fresh CM containing 2.5 μg/ml of ODN 2006 and 5 μM of Chk2 inhibitor II. At this time, ODN 2006 may not be necessary.
B95-8 (marmoset B cell line; University of North Carolina-Chapel Hill)
CD40L-L cells (mouse fibroblasts transfected with human CD40L)
ODN 2006 (Invivogen; tlrl-hodnb-5)
Chk2 inhibitor II (EMD/Calbiochem; 220486)

### RNA Later Protocols

Freezing

In 96-well culture plate, have cells cultured in 100 μl/well supernatant (i.e. remove supernatant in excess, if necessary)

Add 150 μl/well on RNAlater and freeze at −80° C.

Thawing
Keep PBS cold throughout the procedure
Thaw 96-plate and keep it on ice
Transfer cells from wells into RNase-free sterile eppendorf tube (300 μl)
Add sterile PBS up to 1.5 ml to each tube
Spin in microcentrifuge at 300×g for 10 minutes.
Aspirate the buffer and resuspend in 210 μl PBS
Take 10 μl, add 10 μl trypan blue and count at the hemocytometer
Keep cells on ice and perform RNA extraction as soon as possible

Alternatively, cells can be single-cell sorted into PCR plates in wells containing mastermix using FSC and SSC gating (no PI)

### Optimization of Conditions for EBV Transformation of B Cells—Experiment #5 (96-Well Format)

I. Purpose:

To improve Epstein Barr virus (EBV) transformation efficiency and to obtain monoclonal EBV-B cell lines without limiting dilution methods

II. Samples:

Memory B cells isolated from frozen/thawed PBMC by using a customized Miltenyi kit

III. Methods:

Memory B cells were incubated with EBV suspension (B95-8; 1 ml/100,000 B cells) for infection in the presence of ODN2006 (2.5 μg/ml)+Chk2 inhibitor (2-arylbenzamidazole; 5 μM) or IL-2 (1,000 IU/ml)+R848 (2.5 μg/ml). After overnight incubation, the cells were resuspended in the media containing the same concentrations of the above drugs and distributed into 96-well plates (round bottom) at 30 cells/well. The cells were co-cultured with γ-irradiated feeder cells as indicated in the Table III. Two weeks after EBV-infection, each well was examined under a microscope and the number of wells containing a clump of live lymphoblast cell line (LCL) determined to estimate the overall transformation efficiency. In addition, levels of total IgG in the culture supernatants were measured using an IgG-specific immunoglobulin (Ig) ELISA to determine transformation efficiency of IgG-producing B cells.

TABLE I
Treatment protocols

| No. | Treatment |
|---|---|
| 1 | ODN2006 (2.5 μg/ml) + Chk2 inhibitor (5 μM) |
| 2 | IL-2 (1,000 IU/ml*) + R848 (2.5 μg/ml) |

IL-2, 1,000 IU/ml is equivalent to 280 Roche U/ml.

TABLE II
Cell viability after overnight incubation

| Treatment No. | Input cell number | Viable cell number | Viability |
|---|---|---|---|
| 1 | 20,000 | 14,250 | 60.3% |
| 2 | 20,000 | 15,390 | 74.6% |

TABLE III
The γ-irradiated feeder cells

| | Radiation | | Total number of |
|---|---|---|---|
| Feeder cells | dose (Gy) | Cell number/well | wells plated |
| No feeder | N/A | N/A | 60 |
| PBMC (autologous) | 12 | 30,000 | 30 |
| B cell-depleted PBMC | 12 | 30,000 | 210 |
| (autologous) | | | |
| J774A.1 | 40 | 40,000 | 160 |
| CD40L-L | 75 | 5,000 | 160 |
| J774A.1 + CD40L-L | 40/75 | 40,000/5,000 | 160 |

### Optimization of Conditions for EBV Transformation of B Cells Experiment #6 (96-Well Format)

IV. Purpose:

To improve Epstein Barr virus (EBV) transformation efficiency and to obtain monoclonal EBV-B cell lines without limiting dilution methods

V. Samples:

Memory B cells isolated from frozen/thawed PBMC by using a customized Miltenyi kit

VI. Methods:

Memory B cells were incubated with EBV suspension (B95-8; 1 ml/100,000 B cells) for infection in the presence of ODN2006 (2.5 μg/ml)+Chk2 inhibitor (2-arylbenzamidazole; 5 μM) or IL-2 (1,000 IU/ml)+R848 (2.5 μg/ml). After overnight incubation, the cells were resuspended in the media containing the same concentrations of the above drugs and distributed into 96-well plates (round bottom) at 10 or 30 cells/well. The cells were co-cultured with γ-irradiated feeder cells as indicated in the Table VI. Two weeks after EBV-infection, we examined each well under a microscope and determined the number of wells containing a clump of live lymphoblast cell line (LCL) to estimate the overall transformation efficiency. In addition, levels of total IgG in the culture supernatants were measured using an IgG-specific immunoglobulin (Ig) ELISA to determine transformation efficiency of IgG-producing B cells.

TABLE IV
Treatment protocols

| No. | Treatment |
|---|---|
| 1 | ODN2006 (2.5 μg/ml) + Chk2 inhibitor (5 μM) |
| 2 | IL-2 (1,000 IU/ml*) + R848 (2.5 μg/ml) |

IL-2, 1,000 IU/ml is equivalent to 280 Roche U/ml.

TABLE V
Cell viability after overnight incubation

| Treatment No. | Input cell number | Viable cell number | Viability |
|---|---|---|---|
| 1 | 20,000 | 18,800 | 74.4% |
| 2 | 20,000 | 16,200 | 68.9% |

TABLE VI
The γ-irradiated feeder cells

| | Radiation | | Total number of |
|---|---|---|---|
| Feeder cells | dose (Gy) | Cell number/well | wells plated |
| No feeder | N/A | N/A | 80 |
| PBMC (autologous) | 12 | 10,000 | 160 |
| B cell-depleted PBMC | 12 | 10,000 | 40 |
| (autologous) | | | |
| J774A.1 | 40 | 40,000 | 160 |
| CD40L-L | 75 | 5,000 | 160 |
| J774A.1 + CD40L-L | 40/75 | 40,000/5,000 | 160 |

### Experiment 8-28—Total IgG ELISA

| | Frequency | | | | | | |
|---|---|---|---|---|---|---|---|
| Sample | of positive | | | | | CDR3 | |
| ID | PCR wells | V | DH | J | Mutated | length | Productive |
| 1 | 13/20 | 1~69*02 | 3~3*01 | 4*02 | 0.037249 | 9 | F |
| 2 | 11/20 | 4~39*06 | 6~13*01 | 1*01 | 0.043010 | 15 | F |
| | 1/20 | 3~49*03 | 2~2*01/inv, 02/inv | 4*02 | 0.092537 | 2 | N |
| 3 | 4/20 | 3~23*01 | 2~2*01/inv, 02/inv | 3*02 | 0.029491 | 17 | F |
| 4 | 3/20 | 4~39*01 | 2~21*02 | 5*01 | 0.052219 | 19 | F |
| 5 | 3/20 | 2~5*10 | 4~17*01 | 4*02 | 0.041436 | 13 | F |
| 6 | 13/20 | 4~31*03 | 0~IR*01C | 4*02 | 0.021938 | 10 | F |
| 7 | 3/20 | 4~34*01 | 2~2*01, 02 | 4*02 | 0.002667 | 18 | F |
| 8 | 8/20 | 4~59*01 | 2~2*02 | 4*01 | 0.037534 | 18 | F |

| Tube | Original | Sort | | | | | CDR3 | Ig | |
|---|---|---|---|---|---|---|---|---|---|
| ID | Well | Well | V | DH | J | Mutated | length | Isotype | Productive |
| 1 | B7 | A10 | 1~69*02 | 3~3*01 | 4*02 | 0.037463977 | 9 | G1 | F |
| 1 | B7 | A10 | 1~69*02 | 3~3*01 | 4*02 | 0.037249284 | 9 | G1 | F |
| 1 | B7 | A10 | 1~69*02 | 3~3*01 | 4*02 | 0.037356322 | 9 | G1 | F |
| 1 | B7 | A2 | 1~69*02 | 3~3*01 | 4*02 | 0.040114613 | 9 | G1 | F |
| 1 | B7 | A2 | 1~69*02 | 3~3*01 | 4*02 | 0.040114613 | 9 | G1 | F |
| 1 | B7 | A2 | 1~69*02 | 3~3*01 | 4*02 | 0.040229885 | 9 | G1 | F |
| 1 | B7 | A3 | 1~69*02 | 2~2*01, 02 | 1*01 | 0.074498567 | 9 | M | N |
| 1 | B7 | A3 | 1~69*02 | 3~3*01 | 4*02 | 0.037249284 | 9 | G1 | F |
| 1 | B7 | A3 | 1~69*02 | 3~10*01/inv | 1*01 | 0.044247788 | 9 | A1 | N |
| 1 | B7 | A4 | 1~69*02 | 3~3*01 | 4*02 | 0.037249284 | 9 | G1 | F |
| 1 | B7 | A4 | 1~69*02 | 3~3*01 | 4*02 | 0.037249284 | 9 | G1 | F |
| 1 | B7 | A4 | 1~69*02 | 3~3*01 | 4*02 | 0.037356322 | 9 | G1 | F |
| 1 | B7 | A5 | 1~69*02 | 3~3*01 | 4*02 | 0.042979943 | 9 | G1 | F |
| 1 | B7 | A5 | 1~69*02 | 3~3*01 | 4*02 | 0.037249284 | 9 | G1 | F |
| 1 | B7 | A5 | 1~69*02 | 3~3*01 | 4*02 | 0.037356322 | 9 | G1 | F |
| 1 | B7 | A9 | 1~69*02 | 3~3*01 | 4*02 | 0.037249284 | 9 | G1 | F |
| 1 | B7 | A9 | 1~69*02 | 2~2*01, 02 | 1*01 | 0.066666667 | 9 | G1 | N |
| 1 | B7 | A9 | 1~69*02 | 3~3*01 | 4*02 | 0.037356322 | 9 | G1 | F |
| 1 | B7 | B11 | 1~69*02 | 3~3*01 | 4*02 | 0.037249284 | 9 | G1 | F |
| 1 | B7 | B11 | 1~69*02 | 3~3*01 | 4*02 | 0.037249284 | 9 | G1 | F |
| 1 | B7 | B11 | 1~69*02 | 3~3*01 | 4*02 | 0.037356322 | 9 | G1 | F |
| 1 | B7 | B2 | 1~69*02 | 3~3*01 | 4*02 | 0.037249284 | 9 | G1 | F |
| 1 | B7 | B2 | 1~69*02 | 3~3*01 | 4*02 | 0.037249284 | 9 | G1 | F |
| 1 | B7 | B2 | 1~69*02 | 3~3*01 | 4*02 | 0.037356322 | 9 | G1 | F |
| 1 | B7 | B3 | 1~69*02 | 3~3*01 | 4*02 | 0.037249284 | 9 | G1 | F |
| 1 | B7 | B3 | 1~69*02 | 3~3*01 | 4*02 | 0.037249284 | 9 | G1 | F |
| 1 | B7 | B3 | 1~69*02 | 2~2*01, 02 | 1*01 | 0.063037249 | 9 | G1 | N |
| 1 | B7 | B4 | 1~69*02 | 3~3*01 | 4*02 | 0.037249284 | 9 | G1 | F |
| 1 | B7 | B4 | 1~69*02 | 3~3*01 | 4*02 | 0.037249284 | 9 | G1 | F |
| 1 | B7 | B4 | 1~69*02 | 3~3*01 | 4*02 | 0.037356322 | 9 | G1 | F |
| 1 | B7 | B6 | 1~69*02 | 3~3*01 | 4*02 | 0.037249284 | 9 | G1 | F |
| 1 | B7 | B6 | 1~69*02 | 3~3*01 | 4*02 | 0.037249284 | 9 | G1 | F |
| 1 | B7 | B6 | 1~69*02 | 3~3*01 | 4*02 | 0.037356322 | 9 | G1 | F |
| 1 | B7 | B7 | 1~69*02 | 3~3*01 | 4*02 | 0.037249284 | 9 | G1 | F |
| 1 | B7 | B7 | 1~69*02 | 3~3*01 | 4*02 | 0.037249284 | 9 | G1 | F |
| 1 | B7 | B7 | 1~69*02 | 3~3*01 | 4*02 | 0.037356322 | 9 | G1 | F |
| 1 | B7 | B8 | 1~69*02 | 3~3*01 | 4*02 | 0.037249284 | 9 | G1 | F |
| 1 | B7 | B8 | 1~69*02 | 3~3*01 | 4*02 | 0.037249284 | 9 | G1 | F |
| 1 | B7 | B8 | 1~69*02 | 3~3*01 | 4*02 | 0.037356322 | 9 | G1 | F |
| 2 | C5 | C11 | 4~39*06 | 6~13*01 | 1*01 | 0.043010753 | 15 | G1 | F |
| 2 | C5 | C2 | 4~39*06 | 6~13*01 | 1*01 | 0.043010753 | 15 | G1 | F |
| 2 | C5 | C4 | 4~39*06 | 6~13*01 | 1*01 | 0.043010753 | 15 | G1 | F |
| 2 | C5 | C5 | 4~39*06 | 6~13*01 | 1*01 | 0.043010753 | 15 | G1 | F |
| 2 | C5 | C7 | 4~39*06 | 6~13*01 | 1*01 | 0.043010753 | 15 | G1 | F |
| 2 | C5 | C8 | 4~39*06 | 6~13*01 | 1*01 | 0.043010753 | 15 | G1 | F |
| 2 | C5 | C9 | 4~39*06 | 6~13*01 | 1*01 | 0.043010753 | 15 | G1 | F |
| 2 | C5 | D11 | 4~39*06 | 6~13*01 | 1*01 | 0.043010753 | 15 | G1 | F |
| 2 | C5 | D4 | 4~39*06 | 6~13*01 | 1*01 | 0.042895442 | 15 | G1 | F |
| 2 | C5 | D7 | 3~49*03 | 2~2*01, 02 | 4*02 | 0.125401929 | 2 | M | N |
| 2 | C5 | D7 | 3~49*03 | 2~2*01/inv, 02/inv | 4*02 | 0.092261905 | 2 | G1 | N |
| 2 | C5 | D7 | 4~39*06 | 6~13*01 | 1*01 | 0.043010753 | 15 | G1 | F |
| 2 | C5 | D7 | 3~49*03 | 2~2*01/inv, 02/inv | 4*02 | 0.092537313 | 2 | G1 | N |
| 2 | C5 | D9 | 4~39*06 | 6~13*01 | 1*01 | 0.043010753 | 15 | G1 | F |
| 3 | C8 | E2 | 3~23*01 | 2~2*01/inv, 02/inv | 3*02 | 0.029490617 | 17 | G1 | F |
| 3 | C8 | E2 | 3~23*01 | 2~2*01/inv, 02/inv | 3*01 | 0.034852547 | 17 | G1 | F |
| 3 | C8 | E2 | 3~23*01 | 5~24*01 | 3*02 | 0.068181818 | 17 | G4 | N |
| 3 | C8 | E4 | 3~23*01 | 5~24*01 | 3*01 | 0.053619303 | 17 | M | N |
| 3 | C8 | E4 | 3~23*01 | 2~2*01/inv, 02/inv | 3*02 | 0.029569892 | 17 | G1 | F |
| 3 | C8 | E4 | 3~23*01 | 5~24*01 | 3*01 | 0.059139785 | 17 | G2 | N |
| 3 | C8 | E6 | 3~23*01 | 2~2*01/inv, 02/inv | 3*02 | 0.032258065 | 17 | G1 | F |
| 3 | C8 | F9 | 3~23*01 | 2~2*01/inv, 02/inv | 3*02 | 0.029490617 | 17 | G4 | F |
| 3 | C8 | F9 | 3~23*01 | 2~2*01/inv, 02/inv | 3*02 | 0.029490617 | 17 | G1 | F |
| 3 | C8 | F9 | 3~23*01 | 2~2*01/inv, 02/inv | 3*02 | 0.029333333 | 17 | G1 | F |
| 3 | C8 | F9 | 3~23*01 | 2~2*01/inv, 02/inv | 3*02 | 0.029569892 | 17 | G1 | F |
| 4 | D8 | H2 | 4~39*01 | 2~21*02 | 5*01 | 0.052219321 | 19 | G1 | F |
| 4 | D8 | H6 | 4~39*01 | 2~21*02 | 5*01 | 0.052219321 | 19 | G1 | F |
| 4 | D8 | H7 | 4~39*01 | 2~21*02 | 5*01 | 0.052219321 | 19 | G1 | F |
| 5 | D9 | A2 | 2~5*10 | 4~17*01 | 4*02 | 0.041436464 | 13 | G1 | F |
| 5 | D9 | B11 | 2~5*10 | 4~17*01 | 4*02 | 0.041436464 | 13 | G1 | F |
| 5 | D9 | B2 | 2~5*10 | 4~17*01 | 4*02 | 0.041436464 | 13 | G1 | F |
| 6 | E11 | C10 | 4~31*03 | 1~IR1*01 | 4*02 | 0.025210084 | 10 | G1 | N |
| 6 | E11 | C11 | 4~31*03 | 0~IR*01C | 4*02 | 0.022408964 | 10 | G1 | F |
| 6 | E11 | C2 | 4~31*03 | 0~IR*01C | 4*02 | 0.022792023 | 10 | G1 | F |
| 6 | E11 | C3 | 4~31*03 | 0~IR*01C | 4*02 | 0.019073569 | 10 | E | F |
| 6 | E11 | C3 | 4~31*03 | 0~IR*01C | 4*02 | 0.019830028 | 10 | G1 | F |
| 6 | E11 | C3 | 4~31*03 | 0~IR*01C | 4*02 | 0.01971831 | 10 | G1 | F |
| 6 | E11 | C3 | 4~31*03 | 0~IR*01C | 4*02 | 0.019607843 | 10 | G1 | F |
| 6 | E11 | C4 | 4~31*03 | 0~IR*01C | 4*02 | 0.022535211 | 10 | G1 | F |
| 6 | E11 | C4 | 4~31*03 | 0~IR*01C | 4*02 | 0.022408964 | 10 | G1 | F |
| 6 | E11 | C5 | 4~31*03 | 0~IR*01C | 4*02 | 0.022535211 | 10 | G1 | F |
| 6 | E11 | C5 | 4~31*03 | 0~IR*01C | 4*02 | 0.02247191 | 10 | G1 | F |
| 6 | E11 | C6 | 4~31*03 | 0~IR*01C | 4*02 | 0.022408964 | 10 | G1 | F |
| 6 | E11 | C6 | 4~31*03 | 0~IR*01C | 4*02 | 0.024922118 | 10 | D | F |
| 6 | E11 | C7 | 4~31*03 | 0~IR*01C | 4*02 | 0.022408964 | 10 | G1 | F |
| 6 | E11 | C9 | 4~31*03 | 0~IR*01C | 4*02 | 0.022222222 | 10 | G1 | N |
| 6 | E11 | D10 | 4~31*03 | 0~IR*01C | 4*02 | 0.019830028 | 10 | G1 | F |
| 6 | E11 | D10 | 4~31*03 | 0~IR*01C | 4*02 | 0.019607843 | 10 | G1 | F |
| 6 | E11 | D10 | 4~31*03 | 0~IR*01C | 4*02 | 0.019553073 | 10 | G1 | F |
| 6 | E11 | D3 | 4~31*03 | 0~IR*01C | 4*02 | 0.02247191 | 10 | G1 | F |
| 6 | E11 | D4 | 4~31*03 | 0~IR*01C | 4*02 | 0.022346369 | 10 | G1 | F |
| 6 | E11 | D6 | 4~31*03 | 0~IR*01C | 4*02 | 0.02259887 | 10 | G1 | F |
| 6 | E11 | D6 | 4~31*03 | 0~IR*01C | 4*02 | 0.023880597 | 10 | G1 | F |
| 6 | E11 | D9 | 4~31*03 | 0~IR*01C | 4*02 | 0.02266289 | 10 | G1 | F |
| 6 | E11 | D9 | 4~31*03 | 0~IR*01C | 4*02 | 0.02247191 | 10 | G1 | F |
| 6 | E11 | D9 | 4~31*03 | 0~IR*01C | 4*02 | 0.02247191 | 10 | G1 | F |
| 7 | B2 | E10 | 4~34*03 | 2~2*01, 02 | 4*02 | 0.002666667 | 18 | G1 | F |
| 7 | B2 | E4 | 4~34*03 | 2~2*01, 02 | 4*02 | 0.002666667 | 18 | G1 | F |
| 7 | B2 | E8 | 4~34*03 | 2~2*01, 02 | 4*02 | 0.002666667 | 18 | G1 | F |
| 8 | G11 | G3 | 4~59*01 | 2~2*02 | 4*01 | 0.037533512 | 18 | G1 | F |
| 8 | G11 | G4 | 4~59*01 | 2~2*02 | 4*01 | 0.037433155 | 18 | G1 | F |
| 8 | G11 | G6 | 4~59*01 | 2~2*02 | 4*01 | 0.037433155 | 18 | G1 | F |
| 8 | G11 | G7 | 4~59*01 | 2~2*02 | 4*01 | 0.037634409 | 18 | G1 | F |
| 8 | G11 | H10 | 4~59*01 | 2~2*02 | 4*01 | 0.037533512 | 18 | G1 | F |
| 8 | G11 | H3 | 4~59*01 | 2~2*02 | 4*01 | 0.037533512 | 18 | G1 | F |
| 8 | G11 | H5 | 4~59*01 | 2~2*02 | 4*01 | 0.037533512 | 18 | G1 | F |
| 8 | G11 | H9 | 4~59*01 | 2~2*02 | 4*01 | 0.037533512 | 18 | G1 | F |

PCR products from fresh single-cell sorted or bulk RNAlater-treated cultures at
10 cells/well IgG + memory B cells from a chronically HIV-1 infected subject

| Orig- | Single-cell sorts from fresh cultures | | | | | | | Bulk RNAlater-treated culture | | | | | |
|---|---|---|---|---|---|---|---|---|---|---|---|---|---|
| inal | No. of | | | | | CDR3 | Produc- | | | | | CDR3 | Produc- |
| Well | sequences | V | DH | J | Mutated | length | tive | V | DH | J | Mutated | length | tive |
| D9 | 3 | 2~5*10 | 4~17*01 | 4*02 | 0.04144 | 13 | F | 2~5*10 | 4~17*01 | 4*02 | 0.04144 | 13 | F |
| E11 | 1 | 4~31*03 | 1~IR1*01 | 4*02 | 0.02521 | 10 | N | 4~31*03 | 1~IR1*01 | 2*01 | 0.04735 | 10 | N |
| | 24 | 4~31*03 | 0~IR*01C | 4*02 | 0.02241 | 10 | F | 3~30*04 | 3~10*02 | 4*02 | 0.02023 | 8 | N |
| | | | | | | | | 3~30*04 | 3~16*01, 02 | 4*02 | 0.00000 | 8 | F |
| B2 | 3 | 4~34*01 | 2~2*01,02 | 4*02 | 0.00267 | 18 | F | 4~34*01 | 2~2*01, 02 | 4*02 | 0.00267 | 18 | F |
| | | | | | | | | | 2~8*02 | 1*01 | 0.10765 | 10 | N |
| | | | | | | | | 1~2*01 | 1~IR1*01C | 5*01 | 0.22345 | 44 | N |
| C5 | 11 | 4~39*06 | 6~13*01 | 1*01 | 0.04301 | 15 | F | 4~39*06 | 6~13*01 | 1*01 | 0.06971 | 15 | N |
| | 1 | 3~49*03 | 2~2*01, 02 | 4*02 | 0.12540 | 2 | N | 3~49*03 | 2~2*01/inv, | 4*02 | 0.09226 | 2 | N |
| | | | | | | | | | 02/inv | | | | |
| | 2 | 3~49*03 | 2~2*01/inv, | 4*02 | 0.09226 | 2 | N | 3~49*03 | 3~10*02/inv | 2*01 | 0.14080 | 7 | N |
| | | | 02/inv | | | | | | | | | | |
| | | | | | | | | 3~49*01 | 5~12*01/inv | 2*01 | 0.17870 | 8 | N |

### Experiment 8-29—Total IgG ELISA

### Experiment 8-30—Total IgG ELISA

### Experiment 8-31—Total IgG ELISA

### RESULTS

This method was used to isolate broadly neutralizing anti-HIV-1 monoclonal antibodies from a chronically HIV-1 infected subject. The subject was infected with a clade A HIV-1 virus and showed broad serum neutralization. Approximately 20,000,000 peripheral blood mononuclear cells (PBMCs) were collected and approximately 30,000 viable IgG+ memory B cells were obtained after enrichment and overnight EBV transformation. The cells were cultured at a density of 8 cells/well.

At the end of stimulation, the 3,600 cultures were screened for total IgG production using an ELISA assay, clade B transmitted founder gp140 Env63521 binding (ELISA) and neutralization of the difficult-to-neutralize HIV-1 CAP45 strain (tier 3, clade C; TZM-bl assay (Li et al, J. Virol. 80:11776-11790 (2006)). Aliquots of cells from each well were both (1) further expanded for supernatant collection and liquid nitrogen freezing, and (2) froze in RNAlater.

In this experiment, 1799/3600 cultures (50%) secreted IgG, as expected. Of them, 24 cultures bound the gp140 Env63521 (1.33%) and 26 clones neutralized >50% of HIV-1 CAP45 (range: 51.2-85.6%). Only 1 clone that bound gp140 Env 63521 also neutralized HIV-1 CAP45.

Interestingly, three clones that neutralized HIV-1 CAP45 were of the IgA and IgM isotype. This is in line with the notion that there was enrichment for IgG-secreting memory B cells (80 to 90% purity), rather than complete purification.

The VH and VL chains from bulk cells frozen at the day of harvest of the 20 best neutralizers were amplified and it was found that 37% were monoclonal and 52.6% were oligoclonal (2 or 3 functional sequences per culture). On supernatant from cultures expanded for an additional week, a check was made for breadth of neutralization (5 difficult-to-neutralize viruses, including CAP45, and SVA as negative control) and breadth of binding to transmitted founder envelopes (3 envelopes, including Env64521). It was found that 2 clones (1-27-G11 and 1-19-B7) neutralized >50% ¾ of the viruses. Clone 1-27-G11 had only 2 VH chains. VH and VL chains are being expressed in a transfection system to retrieve the monoclonal antibodies. No neutralizing clones bound to the transmitted envelopes whereas 9 of the 24 transmitted gp140 Env63521-binding clones previously identified (37.5%) also bound to Env1086C and Env00MSA

All documents and other information sources cited above are hereby incorporated in their entirety by reference.

What is claimed is:

1. A method of producing immortalized B cells comprising transforming B cells with Epstein Barr Virus (EBV) under conditions such that said immortalization is effected.

2. The method according to claim I wherein said B cells are IgG+ memory B cells.

3. The method according to claim 2 wherein said B cells are obtained from peripheral blood.

4. The method according to claim 1 wherein said B cells are obtained from peripheral blood, mucosal tissue or lymphoid tissue.

5. The method according to claim 1 wherein said B cells IgM+ or IgA-memory B cells.

6. The method according to claim 1 further comprising expanding said immortalized B cells in culture.

7. The method according to claim 6 further comprising isolating cells from said expanded population that produce antibodies having a desired antigen specificity.

8. The method according to claim 7 further comprising cloning said isolated cells and isolating from said cloned cells nucleic acid sequences encoding said antibodies, or the variable heavy or variable light chain thereof.

9. The method according to claim 6 wherein said cells are expanded monoclonally, oligoclonally or polyclonally.

10. The method according to claim 1 wherein said B cells are mammalian B cells.

11. The method according to claim 10 wherein said B cells are human B cells.
